(19) United States
(12) Patent Application Publication
(10) Pub. No.: US 2005/0250127 A1
(43) Pub. Date: Nov. 10, 2005

(54) Methods for identifying modulators of MDA-7 mediated apoptosis

(76) Inventors: Paul B. Fisher, Scarsdale, NY (US); Devanand Sarkar, Elmsford, NY (US); Rahul V. Gopalkrishnan, New York, NY (US); Moira Sauane, New York, NY (US)

Correspondence Address: BAKER & BOTTS, 30 ROCKEFELLER PLAZA, NEW YORK, NY, 10112, US

(21) Appl. No.: 11/019,897

(22) Filed: Dec. 22, 2004

Related U.S. Application Data
(63) Continuation of application No. PCT/US03/21237, filed on Jul. 3, 2003.
(60) Provisional application No. 60/394,185, filed on Jul. 3, 2002.
(60) Provisional application No. 60/453,003, filed on Mar. 7, 2003.

(30) Foreign Application Priority Data
Jul. 3, 2003 (WO) PCT/US03/21237

(51) Int. Cl. C12Q 1/68; G01N 33/574

(52) U.S. Cl. 435/6; 435/7.23

(57) ABSTRACT

The present invention relates to the discoveries that apoptotic effects of the melanoma differentiation associated gene mda-7 (also known as interleukin-24, “IL-24”) on malignant cells occur via the p38 MAPK pathway and members of the Growth Arrest and DNA Damage (“GADD”) gene family but are substantially independent of the JAK/STAT pathway. Accordingly, the invention provides for methods for identifying apoptosis-modulating agents using assay methods which determine the ability of a test agent to increase or decrease expression of constituents of the mda-7 apoptosis pathway, preferably in a JAK/STAT substantially independent manner. Such agents may be small molecules or may be fragments, variants and/or derivatives of native MDA-7.

### GRANT SUPPORT

The subject matter of this application was funded at least in part by National Institutes of Health grants CA35675, CA87170, CA88906, CA97318 and DK52825, Department of Defense grants BC98-0148, DAMD17-98-1-8053 and DAMD-02-1-0041, and United States Public Health Service Grants R01-CA46465 from the National Cancer Institute and RO1-AI36450, RO1-AI43369 and 2T32AI07403 from the National Institute of Allergy and Infectious Diseases, so that the United States Government has certain rights herein.

### INTRODUCTION

The present invention relates to the discoveries that apoptotic effects of the melanoma differentiation associated gene mda-7 (also known as interleukin-24, “IL-24”) on malignant cells occur via the p38 MAPK pathway and members of the Growth Arrest and DNA Damage (“GADD”) gene family but are substantially independent of the JAK/STAT pathway. Accordingly, the invention provides for methods for identifying apoptosis-modulating agents using assay methods which determine the ability of a test agent to increase or decrease expression of constituents of the mda-7 apoptosis pathway, preferably in a JAK/STAT substantially independent manner. Such agents may be small molecules or may be fragments, variants and/or derivatives of native MDA-7.

### BACKGROUND OF THE INVENTION

Abnormalities in differentiation represent a distinctive characteristic and frequent event in many histologically dissimilar cancers (Sachs, 1978, Nature 274, 535-539; Fisher et al., 1985, Pharmacol. Ther. 27, 143-166; Jiang et al., 1994, Mol. Cell. Different. 2, 221-239). Exploiting these defects in tumor cells represents a novel and potentially less toxic form of therapy, ‘differentiation therapy’ (Leszczyniecka et al., 2001, Pharmacol. Ther. 90, 105-156; Waxman, 1996, in Differentiation therapy (Ares-Serono Symposia Publishers, Rome), pp. 1-531). Treatment of human melanoma cells with a combination of fibroblast interferon (IFN-β) and the protein kinase C activator mezerein (MEZ) results in irreversible growth arrest, terminal differentiation and, over time, programmed cell death (apoptosis) (Fisher et al., 1985, Pharmacol. Ther. 27, 143-166; Jiang et al., 1993, Mol. Cell. Different. 1, 41-66). To define the gene expression changes associated with and potentially causative of these profound changes in melanoma cell physiology, various subtraction hybridization strategies have been employed (Jiang and Fisher, 1993, Mol. Cell. Different. 1, 285-299; Kang et al., 1998, Proc. Natl. Acad. Sci. USA. 95, 13788-13793; Jiang et al., 2000, Proc. Natl. Acad. Sci. USA. 97, 12684-12689; Huang et al., 1999, Gene 236, 125-131; Jiang et al., 1995, Oncogene 11, 2477-2486). This search resulted in the identification of mda-7 as a unique gene selectively upregulated during the process of terminal differentiation and irreversible growth arrest in melanoma cells (Jiang et al., 1995, Oncogene 11, 2477-2486). Current data suggests that mda-7 is a new member of the IL-10 subfamily, which now includes IL-19, IL-TIF, AK-155 and IL-20 (Gallagher, et al., 2000, Genes Immun. 1, 442-450; Zhang et al., 2000, J. Biol. Chem. 275, 24436-24443; Xie et al., 2000, J. Biol. Chem. 275, 31335-31339; Kotenko et al., 2001, J. Biol. Chem. 276, 2725-2732). Based on the presence of an IL-10 signature sequence, a 49 amino acid N-terminal signal peptide, physical location in the human genome on chromosome locus 1q32 in an apparent cytokine cluster, including IL-10, IL-19 and IL-20, and its ability to signal through the IL-20R complex, mda-7 has now been classified as IL-24 (Huang et al., 2001, Oncogene 20, 7051-7063; Wang et al., 2002, J. Biol. Chem. 277, 7341-7347).

An intriguing property of mda-7/IL-24 is its ability, when expressed by means of a replication-incompetent adenovirus, Ad.mda-7, to induce apoptosis in many human cancer cell contexts, while sparing normal human cells from toxicity (Su et al., 1998, Proc. Natl. Acad. Sci. USA. 95, 14400-14405). Ad.mda-7 infection of melanoma, breast carcinoma, colon carcinoma, prostate carcinoma, small cell lung carcinoma and pancreatic carcinoma (when used in combination with antisense-K-ras oligonucleotide) culminates in apoptosis (Su et al., 1998, Proc. Natl. Acad. Sci. USA. 95, 14400-14405; Saeki et al., 2000, Gene Ther. 7, 2051-2057; Su et al., 2001, Proc. Natl. Acad. Sci. USA. 98, 10332-10337; Lebedeva et al., 2002, Oncogene 21, 708-718; Madireddi et al., 2000, Adv. Exp. Med. Biol. 465, 239-261). However, normal melanocytes, endothelial cells, mammary and prostate epithelial cells and skin fibroblasts are refractive to Ad.mda-7-induced killing (Su et al., 1998, Proc. Natl. Acad. Sci. USA. 95, 14400-14405; Saeki et al., 2000, Gene Ther. 7, 2051-2057; Lebedeva et al., 2002, Oncogene 21, 708-718; Madireddi et al., 2000, Adv. Exp. Med. Biol. 465, 239-261). Although the apoptosis-inducing effect of mda-7 is well established, the pathway(s) responsible for this cancer-specific apoptosis has not hitherto been elucidated. Moreover, based on selective cancer-specific killing by Ad.mda-7, this gene may prove effective in the gene-based therapy of cancer (Su et al., 1998, Proc. Natl. Acad. Sci. USA. 95, 14400-14405; Saeki et al., 2000, Gene Ther. 7, 2051-2057; Madireddi et al., 2000, Adv. Exp. Med. Biol. 465, 239-261; Mhashilkar et al., 2001, Mol. Med. 7, 271-282).

Growth-arrest and DNA damage-inducible genes were originally isolated from ultraviolet radiation-treated cells and subsequently grouped according to their coordinate regulation by growth arrest and DNA damage (Fornace, et al., 1989, Mol. Cell Biol. 9, 4196-4203). Later these genes were found to be stress response genes that were induced by ultraviolet radiation, chemical carcinogens, starvation, oxidative stress as well as apoptosis-inducing agents such as TNF-α, C-2 ceramide, dimethyl sphingosine, anti-Fas antibody and staurosporine (Hollander et al., 2001, Int. J. Cancer 96, 22-31; Price and Calderwood, 1992, Cancer Res. 52, 3814-3817; Marten et al., 1994, FASEB J. 8, 538-544; Oh-hashi, et al., 2001, Free Rad. Biol. Med. 30, 213-221). There are five members of this family, namely, GADD34, GADD45α, GADD45β, GADD45γ and GADD153, which encode highly acidic nuclear proteins with similar and unusual charge characteristics (Takekawa and Saito, 1998, Cell 95, 521-530; Zhan et al., 1994, Mol. Cell Biol. 14, 2361-2371). GADD34 is a 73-kDa protein that interacts with a diverse array of proteins within the cell (Hollander et al., 1997, J. Biol. Chem. 272, 13731-13737; Connor, et al., 2001, Mol. Cell Biol. 21, 6841-6850; Hasegawa, et al., 2000, Biochem. Biophys. Res. Commun. 267, 593-596; Hasegawa and Isobe, 1999, Biochim. Biophys. Acta 1428, 161-168; Hasegawa, et al., 1999, Biochem. Biophys. Res. Commun. 256, 249-254; Grishin et al., 2001, Proc. Natl. Acad. Sci. USA. 98, 10172-10177; Adler, et al., 1999, Mol. Cell Biol. 19, 7050-7060). Some of these interactions facilitate growth suppression/apoptosis while others indicate its involvement in translation initiation, DNA recombination or repair, mRNA transport and transcriptional regulation. GADD45, a p53-regulated gene, codes for a 21-kDa protein that interacts with the products of two other p53-regulated genes, p21^{WAF1/CIP1/MDA-6} and PCNA (proliferating cell nuclear antigen), and has been implicated in specific aspects of nucleotide excision repair (Smith et al., 1994, Science 266, 1376-1380; Vairapandi et al., 1996, Oncogene 12, 2579-2594). GADD45 also interacts with MTK1 MAPKKK and thus mediates activation of p38 and JNK MAP kinases in response to environrmental stress (Takekawa and Saito, 1998, Cell 95, 521-530). GADD153, also known as CHOP10 (C/EBP-homologous protein), is a transcription factor containing the basic region-leucine zipper domain that heterodimerizes with members of the C/EBP family of transcription factors and interferes with C/EBP-mediated transcription (Ron and Habener, 1992, Genes Dev. 6, 439-45341). In addition, GADD153 itself can enhance gene transcription by binding to a DNA element or by interactions with other transcription factors like AP-1 (Ubeda et al., 1999, Mol. Cell Biol, 19, 7589-7599; Ubeda et al., 1996, Mol. Cell Biol. 16, 1479-1489). Overexpression of each GADD gene causes growth inhibition and/or apoptosis and combined overexpression of the GADD genes leads to synergistic or cooperative antiproliferative effects (Zhan et al., 1994, Mol. Cell Biol. 14, 2361-2371). Subtractive hybridization, which identified mda-7 (Jiang and Fisher, 1993, Mol. Cell. Different. 1, 285-299; Jiang et al., 1995, Oncogene 11, 2477-2486), also identified upregulation of GADD34 during the process of terminal differentiation and growth arrest in human melanoma cells (Jiang et al., 2000, Proc. Natl. Acad. Sci. USA. 97, 12684-12689).

As discussed above, mda-7 has been classified as encoding a putative cytokine based, among other things, on its localization in the IL-10 gene cluster. In general, cytokines transduce signals after cognate receptor binding via a rapid increase in the tyrosine phosphorylation of JAK/STAT complexes, as a prominent early event, which in turn stimulate cells by diverse additional secondary signaling mechanisms and triggering of specific gene expression profiles to regulate cell proliferation, migration, and apoptosis (Darnell et al., 1994, Science 264: 1415-1421; Weber-Nordt et al., 1998, Leuk. Lymphoma 28: 459-467; Kisseleva et al., 2002, Gene 285: 1-24). Experimental evidence documents that the MDA-7/IL-24 protein is secreted from both normal and tumor cells (Su et al., 2001, Proc. Natl. Acad. Sci. U.S.A. 98: 10332-10337; Su et al., 2003, Oncogene 22:1164-1180; Caudell et al., 2002, J. Immunol. 168: 6041-6046; Lebedeva et al., 2002, Oncogene 21: 708-718) and purified MDA-7/IL-24 can bind to IL-20 hetodimeric receptor complex comprising IL-20R1/IL-20R2 and IL-22 complex, comprising IL-22R/IL-20R2, resulting in the activation of STAT signaling pathways (Dumoutier et al., 2001, J. Immunol. 167: 3545-3549; Parrish-Novak et al., 2002, J. Biol. Chem. 277: 47517-47523; Wang et al., 2002, J. Biol. Chem. 277: 7341-7347).

### SUMMARY OF THE INVENTION

The present invention relates to the discovery of a direct relationship between ectopic expression of mda-7 in melanoma cells and induction of GADD-family genes. Specifically, induction of the GADDs has been found to be regulated by p38 MAPK specifically in melanoma cells, and modifying this signaling pathway by agents that block p38 MAPK or GADD expression protected melanoma cells from mda-7 induced apoptosis. Moreover, mda-7-mediated apoptosis was found to be substantially independent of the JAK/STAT pathway.

The present invention provides for methods which identify agents which can promote or, alternatively, inhibit apoptosis by determining the effect of an agent on one or more constituent of the mda-7 apoptosis pathway and/or JAK/STAT. The fidelity of the agent's action may be confirmed by determining the ability of a modulator of another member of the pathway to abrogate the effect of the test agent.

Modulators of apoptosis which may be identified according to the invention include, but are not limited to, small molecules (e.g. as produced by combinatorial chemistry or rational drug design), nucleic acids, peptides, proteins, immunoglobulins (including immunoglobulin fragments, derivatives, etc.), and peptidomimetic compounds. The scope of the invention further encompasses the identification of MDA-7 fragments, variants and/or derivatives thereof as modulators of apoptosis.

Agents identified as apoptosis promoters may be used in methods which inhibit cell proliferation, including methods of inhibiting tumor growth and treating cancers. Agents determined to be apoptosis inhibitors may be used to promote cell proliferation, for example in the context of cell cultures used for research or commercial purposes, or for promoting the viability of cells in tissues in vitro or in vivo.

### BRIEF DESCRIPTION OF THE FIGURES

FIG. 1A-D. Infection with Ad.mda-7 induces the GADD family of genes in melanoma cells but not in normal immortal melanocytes in a time- and dose-dependent manner. A. Melanoma cells (HO-1, WM35, MeWo and FO-1) and immortalized human melanocytes (FM516) were infected with either Ad.vec or with Ad.mda-7 at an m.o.i. of 100 pfu/cell for 3 days. Total RNA was extracted and Northern analysis was performed using the indicated cDNA probes as described in Section 6. B. FO-1 cells were infected with either Ad.vec (100 pfu/cell) or Ad.mda-7 (1, 10 and 100 pfu/cell) and Northern blot analysis was performed as indicated. C. FO-1 cells were infected with either Ad.vec or Ad.mda-7 (100 pfu/cell) for 3 days. Cell lysates were prepared and Western blot analysis was performed using the indicated antibodies as described in Section 6. D. Percentage of cells displaying hypodiploidy (Aₒ), a measure of apoptosis, by FACS analysis 3 days post-infection with an m.o.i. of 100 pfu/cell of Ad.mda-7.

FIG. 2A-E. Treatment with SB203580 inhibits Ad.mda-7-mediated induction of the GADD-family of genes, p38 MAPK phosphorylation and BCL-2 protein downregulation. A. FO-1 cells were infected with either Ad.vec or with Ad.mda-7 (100 pfu/cell) and were treated with either 1 μM SB203580 for 3 days or with different concentrations of SB203580 for 2 days. Total RNA was extracted and Northern blot analysis was performed. B. FO-1 cells were infected with either Ad.vec or Ad.mda-7 (100 pfu/cell) and treated with 1 μM SB203580 for 3 days. Cell lysates were prepared and Western blot analysis was performed. C. FO-1 (left panel) and FM516 (right panel) cells were infected with either Ad.vec or Ad.mda-7 (100 pfu/cell) for 3 days. Cell lysates were prepared and Western blot analysis was performed with anti-phospho-p38 and anti-p38 MAPK antibodies as described in Section 6. D. FO-1 cells were infected with either Ad.vec or Ad.mda-7 (100 pfu/cell) and were treated with 1 μM SB203580 for 3 days. Cell lysates were prepared and Western blot analysis was performed using the indicated antibodies. E. FO-1 cells were infected with either Ad.vec or Ad.mda-7 (100 pfu/cell) and were either treated with 1 μM SB203580 or infected with dominant negative p38 MAPK (Ad.p38DN; 100 pfu/cell) for 3 days. Cell lysates were prepared and Western blot analysis was performed using the indicated antibodies.

FIG. 3. Inhibition of the p38 MAPK pathway protects FO-1 melanoma cells from Ad.mda-7 mediated cell-death. FO-1 cells were infected with either Ad.vec or Ad.mda-7 (100 pfu/cell) and treated with 1 μM SB203580 or infected with Ad.p38DN (100 pfu/cell). Cell viability was measured by MTT assay after 4 days. Cell viability of Ad.vec treated cells was regarded as 1. *: Significant differences from Ad.mda-7 (p<0.0001).

FIG. 4A-C. Inhibition of the p38 MAPK pathway protects cells from Ad.mda-7 mediated apoptosis. A. FO-1 cells were infected with either Ad.vec or Ad.mda-7 (100 pfu/cell) and treated with 1 μM SB203580 for 3 days. DNA was isolated from the cells and fragmentation was analyzed as described in Section 6. B. FO-1 cells were infected with either Ad.vec or with Ad.mda-7 (100 pfu/cell) and treated with 1 μM SB203580 or infected with Ad.p38DN (100 pfu/cell). Cell cycle was analyzed as described in Section 6. C. FO-1 cells were infected with either Ad.vec or with Ad.mda-7 (100 pfu/cell) and treated with 1 μM SB203580 or infected with Ad.p38DN (100 pfu/cell). Percentage of apoptotic cells at day 1 and day 3 post-infection in each group were plotted.

FIG. 5. inhibition of the GADD family of genes protects FO-1 melanoma cells from Ad.mda-7-mediated cell death. FO-1 cells were transfected with the indicated antisense construct either alone or in combination. After 24 h the cells were infected with either Ad.vec (white bars) or Ad.mda-7 (black bars) (100 pfu/cell) for 3 days. Cell viability was measured by MTT assay. Cell viability of Ad.vec treated cells was regarded as 1. Significant differences from Control, Ad.mda-7 #: p<0.05; *: p<0.005; §: p<0.0001.

FIG. 6A-B. A. A hypothetical model of the involvement of the p38 MAPK pathway and the GADD family members of genes in mediating apoptosis in human melanoma cells by Ad.mda-7. B. Overview of the signaling pathways associated with Ad.mda-7 and MDA-7 activity in cancer cells and the immune system. P-indicates phosphorylation, VEGF: vascular endothelial growth factor; TGF-beta: transforming growth factor-beta; iNOS: inducible nitric oxide synthase; PKR: double-stranded RNA-dependent protein kinase R; MAPL: mitogen activated protein kinase; eIF2-alpha: eukaryotic translation initiation factor 2-alpha; STAT: signal transducer and activator of transcription; GADD: growth arrest and DNA damage inducible; PHA: phytohemaglutinin; LPS: lipopolysaccharide; IL: interleukin; TNF-alpha: tumor necrosis factor-alpha; IFN-gamma: interferon gamma; GM-CSF: granulocyte macrophage-colony stimulating factor.

FIG. 7A-B. Effect of protein tyrosine kinase inhibitors on Ad.mda-7 induced apoptosis: A. Cell lines were grown in absence or presence of AG490 (5 μM) Genistein (10 μM) or AG18 (7 μM) after infection with 100 p.f.u./cell of Ad.vector of Ad.mda-7. Cells were analyzed for cell viability by the MTT proliferation assay as described, 5-days after infection. Numbers represent a ratio of specific treatments indicated versus untreated cells. An average of three independent experiments is shown. B. Induction of apoptosis selectively in different cell lines by Ad.mda-7 in presence of different TK inhibitors, Genistein, AG490 and AG18 was deterrnined by calculating percentage of the cells in A₀ fraction using CellQuest software (Becton Dickinson). Apoptotic fraction (A₀) determinations was made on cells infected with 100 p.f.u./cell of Ad.vec or Ad.mda-7, harvested at 48 h after infection was fixed and stained with PI as described in Section 7 for the respective determinations. Analyses were performed on susceptible DU-145 prostate cancer and resistant normal immortalized human melanocytes FM-516; M1 bars indicate the Aₒ fraction.

FIG. 8A-B. Expression pattern of IL-20R1, IL20R2 and IL22R in different cell lines: RT-PCR analysis using primers and PCR conditions described in Section 7 was performed using RNA from different Ad.mda-7 susceptible or resistant cell lines. A. Ethidium bromide stained agarose gels of DNA derived from RT-PCR reactions are shown for a spectrum of human cancer cells including prostate (DU-145), cervical (HeLa), glioblastoma (H4-GBM), melanoma (C8161, HO-1); normal immortalized cells, hTERT immortalized fetal astrocytes (astrocyte) and SV40 T-Ag immortalized melanocytes (FM-516). Rat embryo fibroblast immortalized by human adenovirus type 5 infection and transformed by oncogenic ras (CREF-ras) was used as a negative control. GAPDH primers were used as positive controls for all RT-PCR reactions. B Ethidium bromide stained agarose gels of DNA derived from RT-PCR reactions for IL-20 and IL-22 receptors are shown for a panel of human breast cancer (MCF-7, MDA-MB-231, MDA-MB453, T47D) and pancreatic cancer (AsPC-1, BxPC-3, MIA PaCa-2 and PANC-1) and immortalized normal breast epithelial cells (HBL-100). Integrity of original RNA was determined by performing PCR with human EF-1α specific primers.

FIG. 9A-B. Expression pattern of IL-20R1, IL20R2 and IL22R in AsPC-1 cell line after different treatment protocols: A. Ethidium bromide stained agarose gels of DNA derived from RT-PCR reactions are shown for AsPC-1 human pancreatic cancer cell line derived RNA with IL-20 and IL-22 receptor specific primers and EF-1alpha positive control. RNA was isolated from uninfected, and Adenoviruses expressing no gene, (Ad.vector), an anti-sense oriented K-ras gene Ad.K-rasAS, mda-7/IL-24 cDNA (Ad.mda-7) or combination of anti-sense K-ras and mda-7 (Ad.mda-7+Ad.K-rasAS). B. Similar RT-PCR reactions were performed in parallel with RNA isolated concurrently with the AsPC-1 samples using DU-145, FM-516 and CREF-ras cells as positive and negative controls respectively.

FIG. 10. Expression pattern of IL-20R1, IL-20R2 and IL22R in human fibrosarcoma cells mutated for components of the JAK/STAT pathway: Ethidium bromide stained agarose gels of DNA derived from RT-PCR reactions are shown corresponding to parental 2f TGH fibrosarcoma and corresponding mutants for IL-20 and IL-22 receptors. Positive and negative controls for each reaction are shown in the HO-1 (human melanoma) and AsPC-1 (pancreatic cancer) lanes respectively.

FIG. 11. Apoptotic activity after Ad.mda-7 infection in JAK/STAT deficient cell lines: Cell line indicated at the top of each panel was incubated in absence or presence of AG490 (5 μM) Genistein (10 μM) or AG18 (7 μM) after infection with 150 p.f.u./cell of Ad.vector or Ad.mda-7. Cells were analyzed for cell viability by MT assay 5 days after infection. MTT absorbance of untreated control cells was set at 1 to determine relative number of viable cells. Results shown are an average of three independent experiments.

FIG. 12A-B. Effect of p38^{MAPK} inhibitor on Ad.mda-7 induced killing in different cell lines: A. Different cell lines were incubated in absence or presence of SB203580 (2 μM) after infection with 100 p.f.u./cell of Ad.vector of Ad.mda-7. Cells were analyzed for cell viability by MTT assay 5-days after infection. MTT absorbance of untreated control cells was set at 1 to determine relative number of viable cells. Results shown are an average of three independent experiments. B. Induction of apoptosis selectively in different cell lines by Ad.mda-7 in presence of SB203580 was determined by calculating percentage of the cells in A₀ fraction using CellQuest software (Becton Dickinson). Apoptotic fraction (A₀) determinations was made on cells infected with 100 p.f.u./cell of Ad.vec or Ad.mda-7, harvested at 48 h after infection was fixed and stained with PI as described in Section 7 for the respective determinations. Analyses were performed on susceptible DU-145 prostate cancer and resistant normal immortalized human melanocytes FM-516; M1 bars indicate the Aₒ fraction.

FIG. 13A-B. Effect of p38^{MAPK} inhibitor and GADD family of gene expression on Ad.mda-7 induced killing in 2f TGH series of cell lines: A. Cell lines were incubated in absence or presence of SB203580 (2 μM) after infection with 150 p.f.u./cell of Ad.vector of Ad.mda-7. Cells were analyzed for cell viability by MTT assay 6-days after infection. MTT absorbance of untreated control cells was set at 1 to determine relative number of viable cells. Results shown are an average of three independent experiments. B. Cell lines were infected either with Ad.vector or Ad.mda-7 at a multiplicity of infection of 150 p.f.u. per cell for 4 days. Total RNA was extracted and Northern analysis was performed by using the indicated cDNA probes as described in Section 7.

### DETAILED DESCRIPTION OF THE INVENTION

The present invention relates to molecules involved in mda-7 mediated apoptosis. It is based, at least in part, on the discoveries that mda-7, introduced into cancer cells, promotes phosphbrylation of p38 MAPK and HSP27, increases expression of members of the GADD family, and decreases levels of anti-apoptotic BCL-2, and further that apoptosis induction by mda-7 has been found to be substantially independent of the JAK/STAT pathway.

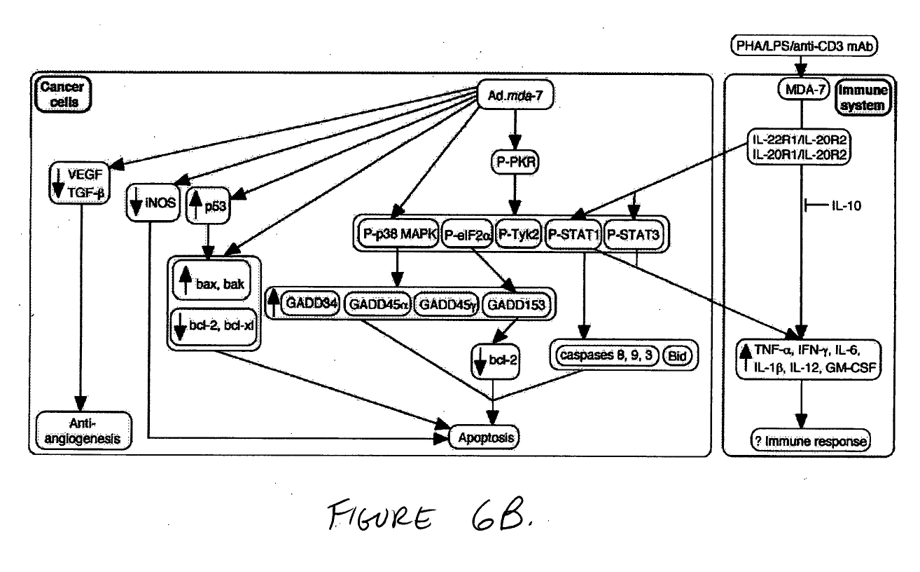

In one aspect, the present invention provides for methods of identifying agents that modulate—promote or alternatively inhibit—apoptosis by assaying the ability of a test agent to alter (i) the phosphorylation of p38 MAPK or HSP27, (ii) expression of one or more member of the GADD family (for example, but not limited to, GADD153, GADD34, GADD45-alpha, GADD45-gamma), and/or (iii) expression of BCL-2. In particular, an agent which is determined to increase phosphorylation of p38 or HSP27, increase expression of one or more member of the GADD family, and decrease expression of BCL-2 may be considered to be an agent that promotes apoptosis via the mda-7 pathway. However, the invention also provides for the identification of apoptosis-modulating agents which may alter one or more constituents of the pathway in a manner inconsistent with the mda-7 pathway. Conversely, an agent which is determined to decrease phosphorylation of p38 or HSP27, decrease expression of one or more member of the GADD family, and increase expression of BCL-2 may be considered to be an agent that does not promote apoptosis via the mda-7 pathway, and likely may not promote apoptosis at all, and may potentially inhibit apoptosis. Without being bound by theory, FIG. 6A depicts a hypothetical model of the mda-7 apoptosis pathway, and FIG. 6B shows a hypothetical scheme by which the mda-7 apoptosis induction pathway may interact with the MDA-7 cytokine pathway and other pathways, for example the anti-angiogenesis pathway.

The ability of an agent to act as a modulator of the mda-7 apoptosis pathway may be tested and corroborated by determining whether an effect produced by the test agent on a first constituent of the pathway can be enhanced or inhibited by a second agent that is an agonist or antagonist of a second constituent of the pathway. For example, the ability of a test agent to increase expression of GADD153 may be evaluated in the presence of an antagonist of p38 MAPK activity, for example, a selective p38 MAPK inhibitor such as SB203580 or in the presence of a dominant negative p38 MAPK mutant, such as AdCMV-Flag38(AGF) (see below). If the test agent acts via the mda-7 pathway, the increase in GADD153 promoted by the agent may be decreased or eliminated by the p38 MAPK antagonist. Similarly, the ability of a test agent to decrease BCL-2 expression may be evaluated in the presence of an antagonist of GADD expression, for example, one or more species of GADD antisense RNA. If the test agent acts via the mda-7 pathway, the decrease in BCL-2 effected by the agent may be decreased or eliminated by the antisense molecules. The consequences of the agonist or antagonist are impacted by the relative positions of the first and second constituent in the pathway. For example, whereas an antagonist of p38 MAPK may inhibit an increase in GADD and/or a decrease in BCL-2, an antagonist of GADD may not inhibit phosphorylation of p38 MAPK, because of what is believed to be their relative positions in the pathway.

In another aspect of the invention, a cell may be determined to be undergoing apoptosis along the mda-7 pathway by detecting, in the cell, increased phosphorylation of p38 MAPK and/or HSP27, increased expression of one or more member of the GADD family, and/or decreased levels of BCL-2 expression. Such methods may be used to either assess the success of an apoptosis modulating treatment (for example, to evaluate the success of apoptosis induction in the treatment of a malignancy) or as corroboration of the effectiveness of a test agent in modulating apoptosis. As such, the methods of the invention may be practiced in a cell in a tumor, for example, in a cell in a biopsy of tumor tissue. Alternatively, the methods of the invention may be practiced in cell culture, or in a viable human or non-human animal.

Assays according to the invention may be performed using one or more cells as a source of members of the mda-7 apoptosis pathway. A cell used according to the invention is preferably a malignant cell, for example, but not by way of limitation, a melanoma cell, a pancreatic cancer cell, a breast cancer cell, a prostate cancer cell, a lung cancer cell, a colon cancer cell, a glioblastoma cell, a lymphoma cell, a leukemia cell, and/or a cell which develops apoptotic features in response to overexpression (increased expression, forced expression) of mda-7 which may, for example, be caused by introduction of an exogenous mda-7 gene and/or exposure to inducing agent(s) such as interferon beta (IFN-beta) and a protein kinase C activator such as mezerein . Preferably, a cell culture is used as a source of cells. However, other sources, for example, a cell present in a human or non-human animal may be used. Where a cell carries an activating mutation in ras, in specific, non-limiting embodiments, an inhibitor of ras may be administered to the cell, for example as set forth in International Patent Application No. PCT/US02/26454, publication no. WO 0316499, incorporated by reference herein. Where a cell is used in a method for identifying an apoptosis-modulating agent, it may be referred to as a “test cell”.

Methods of measuring phosphorylation levels of p38 MAPK and HSP 27, expression of RNA and protein, and detecting and/or quantifying apoptosis and/or cell viability, are well known in the art; some of these techniques are exemplified in the following working examples. Non-limiting examples of methods of detecting/quantifying apoptosis include, but are not limited to, DNA fragmentation analysis, flow cytometry and transferase (TdT)-mediated dUTP nick end labeling (“TUNEL”) assays, and cell viability assessments such as the MTT assay or vital staining.

Modulators of apoptosis which may be identified according to the invention include, but are not limited to, small molecules (e.g. as produced by combinatorial chemistry or rational drug design, preferably, but not by way of limitation, having a molecular size of 500-2000 daltons), nucleic acids, peptides, proteins, immunoglobulins (including immunoglobulin fragments, derivatives, etc.), and peptidomimetic compounds. The scope of the invention further encompasses the identification of MDA-7 variants as inducers of apoptosis (see below). When subjected to the assay methods of the invention, potential modulators are referred to as “test agents”.

The term “MDA-7” as used herein refers to a protein having essentially the amino acid sequence set forth as Genbank Accession Number U16261. A nucleic acid encoding MDA-7 (referred to as “mda-7”) has a sequence as set forth in Genbank Accession No. U16261, or another sequence which, when translated, produces a protein having essentially the same amino acid sequence. See International patent Application No. PCT/US02/26454.

A “mda-7” variant nucleic acid is a nucleic acid encoding a protein which does not have the amino acid sequence set forth as Genbank Accession No. U16261, but which hybridizes to nucleic acid having a sequence as set forth in Genbank Accession No. U16261 under stringent conditions and which, preferably but not by way of limitation, when introduced into a cancer cell such as a melanoma cell inhibits cell proliferation and/or promotes cell death, for example through apoptosis. A specific, non-limiting example of stringent conditions for detecting hybridization of nucleic acid molecules are as set forth in “Current Protocols in Molecular Biology”, Volume I. Ausubel et al., eds. John Wiley:New York N.Y., pp. 2.10.1-2.10.16, first published in 1989 but with annual updating, wherein maximum hybridization specificity for DNA samples immobilized on nitrocellulose filters may be achieved through the use of repeated washings of a hybridized filter in a solution comprising 0.1-2×SSC (15-30 mM NaCl, 1.5-3 mM sodium citrate, pH 7.0) and 0.1% SDS (sodium dodecylsulfate) at temperatures of 65-68 degrees C. or greater. For DNA samples immobilized on nylon filters, a stringent hybridization washing solution may be comprised of 40 mM NaPO₄, pH 7.2, 1-2% SDS and 1 mM EDTA. Again, a washing temperature of at least 65-68 degrees C. is recommended, but the optimal temperature required for a truly stringent wash will depend on the length of the nucleic acid probe, its GC content, the concentration of monovalent cations and the percentage of formamide, if any, that was contained in the hybridization solution.

A MDA-7 variant protein is a protein encoded by a mda-7 variant nucleic acid or a protein which competes with native MDA-7 for binding to an antibody that specifically recognizes native MDA-7.

The present invention further encompasses identifying nucleic acids and proteins which share at least 50 percent, 60 percent, 70 percent, 80 percent, or 90 percent sequence identity with mda-7 or MDA-7 but which do not qualify as variants of the native gene or protein, defined above, as apoptosis-modulating agents. Such molecules are contemplated as potential inducers or inhibitors of apoptosis.

The present invention also encompasses using derivatives of MDA-7, peptide fragments of MDA-7, or derivatized peptide fragments of MDA-7 as test agents to identify, among such molecules, modulators of apoptosis. The term “derivative” or “derivatization” as used herein refers to either chemical modification, for example by the addition of carbohydrate, polyethyleneglycol (“PEG”), lipid, or other functionalities, or the preparation of chimeric, recombinant “fusion” molecules (e.g. a MDA-7 derived fusion protein or fusion peptide). Peptides, and derivatives thereof, preferably contain 5-200, 5-100, 5-50 or 5-20 amino acid residues.

Preferably, and not by way of limitation, “inducing apoptosis” means increasing the level of one or more marker of apoptosis or the percentage of apoptotic cells by at least 10, 20, 30, 40, 50, 60, 70, 80 or 90 percent relative to control values. Conversely, “inhibiting apoptosis” means decreasing the level of one or more marker of apoptosis or the percentage of apoptotic cells, or increasing the percentage of viable cells, by at least 10, 20, 30, 40, 50, 60, 70, 80 or 90 percent relative to control values.

In particular, non-limiting embodiments, the present invention provides for methods for identifying an apoptosis modulating agent (inducer or inhibitor), comprising (a) optionally identifying, as a test cell, a cell which undergoes apoptosis in response to increased expression of a mda-7 gene (caused, for example, by introduction and expression of a mda-7 gene or by induction of an endogenous gene, for example using IFN-beta and a protein kinase C activator such as mezerein), where the test cell may be propagated as a substantially homogenous population of test cells; (b) administering, to a test cell, a test agent; and (c) determining whether there is a change in the level of phosphorylation of p38 MAPK and/or HSP 27 in response to administration of the test agent in the test cell; wherein a change in the level of phosphorylation of p38 MAPK and/or HSP27 indicates that the test agent is a modulator of apoptosis, an increase in phosphorylation being characteristic of an inducer of apoptosis and a decrease in phosphorylation being characteristic of an inhibitor of apoptosis. Such methods may further or alternatively comprise the step of determining whether there is a change in the expression of one or more GADD protein (measured, for example, via promoter activity, RNA and/or protein levels), such asGADD153, GADD34, GADD45-alpha. or GADD45-gamma, wherein an increase in the expression of one or more GADD protein indicates that the test agent is an inducer of apoptosis and a decrease in expression of one or more GADD protein indicates that the test agent is an inhibitor of apoptosis. Any of the foregoing methods may still further comprise a determination of whether the test agent changes the level of expression of BCL-2 (measured, for example, by promoter activity, RNA, or protein level), wherein a decrease is consistent with apoptosis-inducing activity and an increase is consistent with apoptosis-inhibiting activity).

In preferred non-limiting embodiments, a change in the level of phosphorylation or expression means an increase or decrease by at least 10, 20, 30, 40, 50, 60, 70 or 90 percent relative to control values.

In additional embodiments, the present invention provides for methods for identifying an apoptosis inducing agent, comprising administering, to a test cell, a test agent; and determining whether there is, in the test cell, an increase in the level of phosphorylation of a molecule selected from the group consisting of p38 MAPK and HSP 27 in response to administration of the test agent and/or an increase in the level of expression of one or more GADD protein selected from GADD 153, GADD34, GADD45-alpha. or GADD45-gamma in response to administration of the test agent; and optionally further determining whether any increase in phosphorylation of p38 MAPK and/or HSP 27 and/or increase in the level of expression of one or more GADD protein depend upon the operation of the JAK/STAT pathway; wherein an increase in the phosphorylation of p38 MAPK and/or HSP 27 and/or an increase in the level of expression of one or more GADD protein, which are preferably substantially independent of the JAK/STAT pathway, indicate that the test agent is an inducer of apoptosis. Such methods are particularly useful for identifying agents that induce apoptosis via the mda-7 pathway, for example for identifying useful variants of mda-7 nucleic acid or MDA-7 protein.

Section 7 below provides non-limiting examples of methods by which independence of the JAK/STAT pathway may be demonstrated. For example, test cell, in the presence of test agent, can be exposed to (a) a tyrosine kinase inhibitor (such as, but not limited to) genistein or AG18 and/or (b) a JAK-selective inhibitor such as AG490, and/or the test agent's ability to modulate a member of the mda-7 pathway may be determined in a test cell defective in JAK/STAT such as 2f TGH, U1A, U3A, U4A, U5A, or PC-3 cell lines, or a cell which lacks or is deficient in IL-20/IL-22 receptors. If a modulation of a member of the mda-7 pathway by a test agent is substantially unchanged by disruption of or absence of the JAK/STAT pathway, then the operation of the test agent on the pathway member is considered to be independent of the pathway. “Substantially unchanged” in this context means that the effect of test agent on a member of the mda-7 pathway is changed by less than about 30 percent, preferably by less than about 20 percent, by abrogation of JAK/STAT, and may be essentially unchanged.

The foregoing assays may firther optionally be practiced in the presence of one or more additional bioactive agent, including, but not limited to, overexpressed mda-7, X-ray or UV irradiation, a proliferative agent, an antiproliferative agent, etc. Where the assay is performed in conjunction with overexpression of the mda-7 gene, an increase or decrease in the amount of phosphorylation of p38 and/or HSP 27, and/or the expression level(s) of one or more GADD protein and/or BCL-2, may be determined to assess the ability of the test agent to enhance or inhibit mda-7 induced apoptosis.

An apoptosis inducer identified according to the present invention may be used, for example, as an alternative or adjunct to mda-7 gene therapy in a subject in need of such treatment. For example, such an apoptosis inducer may be used in the treatment of cancer in a subject, where the cancer may be, for example, and not by way of limitation, melanoma, glioblastoma multiforme, osteosarcoma, breast cancer, cervical cancer, pancreatic cancer, gastric cancer, hepatoblastoma, colon cancer, lung cancer (adenocarcinoma, small cell, non-small cell, mesothelioma), nasopharyngeal cancer, ovarian cancer, testicular cancer, prostate cancer, leukemia, Hodgkin's lymphoma, non-Hodgkin's lymphoma, bladder cancer, renal carcinoma, etc. Such an apoptosis inducer may selectively exert its effects in malignant cells, as does mda-7, or it may lack such selectivity. Apoptosis inducers which function in non-malignant cells may be used, for example, in tissue ablation or in the treatment of non-malignant hyperproliferative disorders. Effective amounts for particular tumors and patients may be determined by, for example, producing dose response curves.

### EXAMPLE 1

Materials and Methods

Cell Lines, Reagents and Cell Growth Assays. Normal immortal human melanocyte (FM516-SV; FM516), WM35 early radial growth phase (RGP) primary human melanoma and HO-1, FO-1 and MeWo metastatic melanoma cell lines were cultured as previously described (Kerbel et al., 1984, J. Natl. Cancer Inst. 72, 93-108; Fisher et al., 1985, J. Interferon Res. 5, 11-22; Herlyn,1990. Cancer Metastasis Rev. 9, 101-112; Melber et al., 1989, Cancer Res. 49, 3650-3655). SB203580 was purchased from Sigma (St Louis, Mo.). Cell growth and viable cell numbers were monitored by 3-(4,5-dimethylthiazol-2-yl)-2,5-diphenyl tetrazolium bromide (MTT) staining as described in Lebedeva et al., 2000, Cancer Res. 60, 6052-6060.

Virus Construction and Infection Protocol. The construction and purification of mda-7 expressing replication-defective Ad.mda-7 were described in Lebedeva et al., 2002, Oncogene 21, 708-718. The empty adenoviral vector (Ad.vec) was used as a control. A dominant negative kinase-deficient mutant p38 MAPK expressing virus [Flag-p38(AGF)] was prepared by cloning the kinase-inactive p38-alpha (AGF) cDNA (Raingeaud et al., 1995, J. Biol. Chem. 270, 7420-7426; Taher et al., 1999, Biochemistry 38, 13055-13062) into an adenovirus transfer plasmid and made into a replication-incompetent Ad-5 (delE1, E3) virus as described in Valerie, 1999, in Biopharmaceutical Drug Design and Development, eds. Wu-Pong, S. & Rojanasakul, Y (Humana Press, Inc.), pp. 69-142 and Rosenberg et al., 2001, Cancer Res. 61, 764-770. The virus expresses p38alpha (T₁₈₀GY-AGF) with a Flag-tag at its amino terminus. Viral infections were performed as previously described in Lebedeva et al., 2002, Oncogene 21, 708-718.

Plasmid Construction and Transfection Assays. GADD153, GADD34, GADD45alpha and GADD45gamma coding sequences were amplified by RT-PCR. The sequences were verified and ligated into pcDNA3.1(+) (Invitrogen, Carlsbad, Calif.) vector in an anti-sense manner. Plasmids were purified by Qiagen Plasmid Purification Maxi Kit (Qiagen, Hilden, Germany). The day before transfection, 2×104 cells were plated into each well of a 96-well plate. Transient transfection was performed using Maxfect transfection reagent (Molecular Probes International LLC, Eugene, Oreg.) according to the manufacturer's instructions with 500 ng of DNA per well. The total DNA concentration was kept constant by the addition of empty vector.

RNA Isolation and Northern Blot Analysis. Total RNA was extracted from the cells using Qiagen RNeasy mini kit (Qiagen) according to the manufacturer's protocol and Northern blotting was performed as described in Su et al., 1997, Proc. Natl. Acad. Sci. USA. 94, 9125-9130. The cDNA probes used were full length human GADD153, full length human GADD45-alpha, beta and gamma, a 500-bp fragment from human GADD34 and full-length human GAPDH.

Western Blot Analysis. Western blotting was performed as previously described in Lebedeva et al., 2002, Oncogene 21, 708-718. The primary antibodies included: GADD153 (1:500; rabbit polyclonal; Santa Cruz, Santa Cruz, Calif.), GADD34 (1:200; rabbit polyclonal; Santa Cruz), GADD45 (1:200; mouse monoclonal; Santa Cruz), bcl-2 (1:1000; rabbit polyclonal; kindly provided by Dr. John Reed) and EF1 (1:1000; mouse monoclonal; Upstate Biotechnology, Waltham, Mass.).

Phosphorylation of p38-MAPK and HSP27. Cells were harvested in RIPA buffer containing protease inhibitor cocktail, 1 mM Na₃VO₄ and 50 mM NaF and centrifuged at 12,000 rpm for 10 min at 4° C. The supernatant was used as total cell lysate. p38-MAPK was immunoprecipitated from 500 μg of the total cell lysate overnight at 4° C. using 2 μl of rabbit polyclonal anti-p38-MAPK antibody (Calbiochem, San Diego, Calif.). After addition of protein A-agarose incubation was continued for 2 h.

Immunocomplexes were washed 4 times in RIPA buffer, resuspended in 1× SDS-PAGE lysis buffer and transferred to a nitrocellulose membrane. The expressions of phospho-p38-MAPK and total p38-MAPK were detected by Western blot analysis using a rabbit polyclonal anti-phospho-p38-MAPK antibody (New England Biolabs, Beverly, Mass.) and anti-p38-MAPK antibody (Calbiochem, San Diego, Calif.), respectively. The expressions of phospho-HSP27 and total HSP27 in total cell lysate were detected by Western blot analysis using a rabbit polyclonal anti-phospho-HSP27 antibody and a mouse monoclonal anti-HSP27 antibody, respectively (New England Biolabs).

DNA fragmentation assay. Adherent and floating cells from a 10-cm dish were used for DNA fragmentation assays which were performed as previously described in Lebedeva et al., 2002, Oncogene 21, 708-718.

Cell cycle analysis. Cells were harvested, washed in PBS and fixed overnight at −20° C. in 70% ethanol. The cells were treated with RNase A (1 mg/ml) at 37° C. for 30 min and then with propidium iodide (50 μg/ml). Cell cycle was analyzed using a FACScan flow cytometer and data were analyzed using CellQuest software (Becton Dickinson, San Jose, Calif.).

Statistical analysis. Statistical analysis was carried out using one-way analysis of variance (ANOVA), followed by Fisher's protected least significant difference analysis.

Results

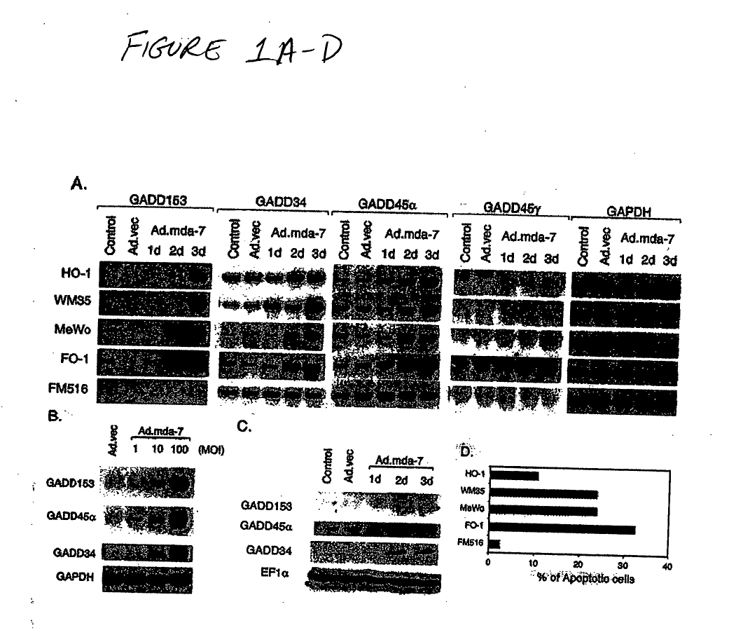

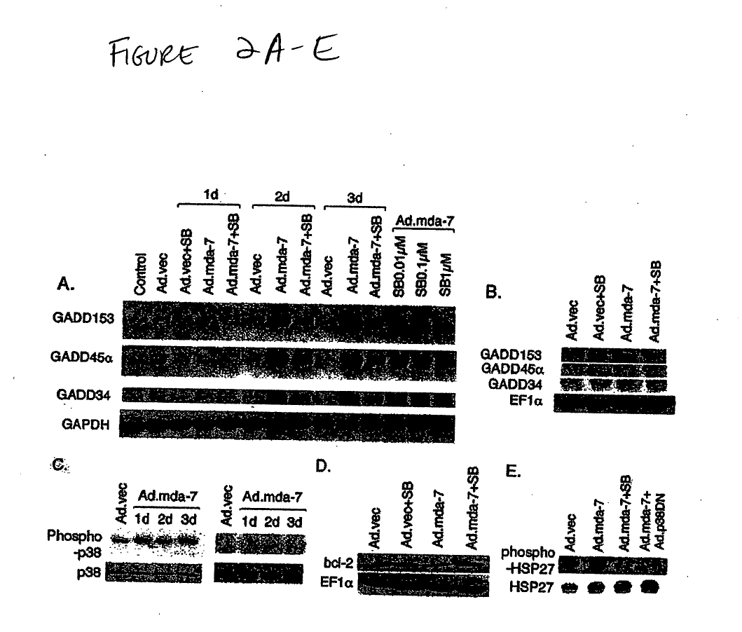

Infection of Human Melanoma Cells, but not Normal Immortal Melanocytes, with Ad.mda-7 Induces the GADD Family of Genes. Melanoma cell lines were infected with either Ad.mda-7 or Ad.vec, total RNA was extracted and the expression pattern of mRNAs of the GADD family of genes was analyzed by Northern blot analysis. As shown in FIG. 1A, the expression of mRNAs for GADD153, GADD34, GADD45alpha and GADD45gamma was either undetectable or low in the control and Ad.vec infected melanoma cells. At day 1 after infection with Ad.mda-7, when there is no effect on the growth of these melanoma cells (Lebedeva et al., 2002, Oncogene 21, 708-718), no change in GADD gene expression was apparent. However, at days 2 and 3 post-infection, there was marked induction of GADD153, GADD34, GADD45alpha and GADD45gamma genes in melanoma cells but not in FM516-SV (FM516) cells, which are immortalized normal human melanocytes (Melber et al., 1989, Cancer Res. 49, 3650-3655). This induction correlates with previous studies indicating that melanoma cells begin dying from 2-days post-infection and infection with Ad.mda-7 causes selective apoptosis of melanoma cells but not of normal melanocytes (FIG. 1D) (Lebedeva et al., 2002, Oncogene 21, 708-718). Among the melanoma cell lines, HO-1 was the most refractory to Ad.mda-7 induction of apoptosis (as measured by increases in the percentage of Aₒ cells) and this correlated with a smaller induction of the GADD family of genes. The other three cell lines, FO-1, MeWo and WM35, were readily killed by Ad.mda-7 (FIG. 1D) (Lebedeva et al., 2002, Oncogene 21, 708-718) and displayed significant induction of the GADD family of genes. Among these three melanoma cell lines FO-1 was the most sensitive to Ad.mda-7 and showed the maximum induction of the GADD family of genes. The expression level of GADD45gamma MRNA was minimal in all the cells and the induction level was also not as significant as it was for the other three members of this family. It should be noted that for GADD45gamma autoradiography was carried out for one week while for the other mRNAs it was performed overnight. No expression of GADD45beta could be detected by Northern blot analysis in any of the control or Ad.mda-7 infected cells. However, RT-PCR analysis detected GADD45beta mRNA, but its level did not change following Ad.mda-7 infection. The expression level of the housekeeping gene GAPDH did not change in any of the cell lines following Ad.mda-7 infection.

Studies were conducted with the FO-1 cell line to investigate further the relationship between Ad.mda-7 infection and induction of the GADD-family of genes. Ad.mda-7 infection of FO-1 cells for 3 days resulted in a dose-dependent induction of the GADD family of genes (FIG. 1B). There was a gradual increase in the level of induction of the GADD family mRNA when cells were infected with an m.o.i. of 1, 10 or 100 pfu/cell. Infection of FO-1 with 100 pfu/cell of Ad.mda-7 also resulted in an increase in the protein levels of the respective GADD family of genes (FIG. 1C). Since no antibody targeting GADD45gamma is currently available, protein expression of this GADD family member was not determined. The level of the housekeeping protein EF1alpha was unchanged following Ad.mda-7 infection documenting the specificity of the induction of the GADD family gene members.

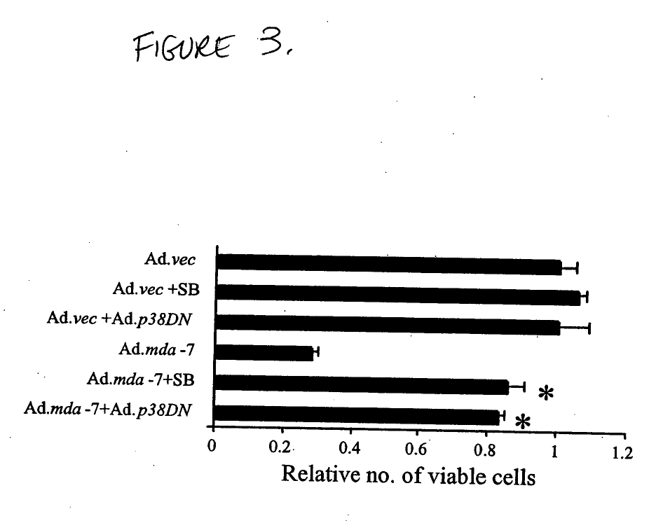

Induction of the GADD Family of Genes by Ad.mda-7 Proceeds Through the p38 MAPK Pathway. Experiments were performed to define the signaling pathway(s) involved in GADD family gene expression following infection with Ad.mda-7. The GADD family of genes is induced by diverse stimuli and one major pathway that is involved in this induction is via the p38 MAPK pathway (Oh-hashi, et al., 2001, Free Rad. Biol. Med. 30, 213-221; Kultz et al., 1998, J. Biol. Chem. 273, 13645-13651; Brenner et al., 1997, J. Biol. Chem. 272, 22173-22181). Since activation of the p38 MAPK pathway results in apoptosis in various cell types, the involvement of this pathway in Ad.mda-7 mediated induction of GADD family of genes was examined. FO-1 cells were infected with Ad.mda-7 and then the cells were treated with SB203580, a selective p38 MAPK inhibitor, to determine effects on induction of the GADD family of genes. SB203580 was also chosen for these studies since this inhibitor was found to selectively block Ad.mda-7 mediated cell death in several cancer cell types, besides melanoma cells. Dose-response studies indicated that the most effective SB203580 concentration, without inducing toxicity, in FO-1 cells was 1 μM. Treatment with SB203580 alone did not affect the basal expression of any of the GADD family of genes (FIG. 2A). However, SB203580 significantly inhibited Ad.mda-7-mediated induction of these genes at days 2 and 3. The inhibitory effect of SB203580 was dose-dependent with minimum inhibition at 0.01 μM and maximum inhibition at 1 μM (FIG. 2A). Western blotting also revealed that SB203580 could inhibit Ad.mda-7-mediated induction of GADD153, GADD34 and GADD45alpha proteins (FIG. 2B).

The effect of Ad.mda-7 infection on p38 MAPK phosphorylation was tested (FIG. 2C). In FO-1 cells, the level of phospho-p38 MAPK increased significantly from day 1 to 3 following Ad.mda-7 infection (FIG. 2C). In contrast, Ad.mda-7 infection of FM516 cells did not induce p38 MAPK phosphorylation. In FO-1 cells and normal immortal melanocytes, equal amounts of total p38 MAPK was detected in Ad.vec and Ad.mda-7 infected cells. The activation of p38 MAPK pathway results in phosphorylation of heat shock protein (HSP) 27 via MAPKAP kinase 2 (Rouse et al., 1994, Cell 78, 1027-1037). To check whether phosphorylation of p38 MAPK by Ad.mda-7 also results in the phosphorylation of this downstream target, the expression levels of phospho-HSP27 and total HSP27 in FO-1 cells were determined following Ad.mda-7 infection. Ad.mda-7 infection resulted in phosphorylation of HSP27 (FIG. 2E) and this phosphorylation could be blocked by treatment with SB203580 or by infection with a replication incompetent adenovirus expressing a dominant negative p38 MAPK (Raingeaud et al., 1995, J. Biol. Chem. 270, 7420-7426; Taher et al., 1999, Biochemistry 38, 13055-13062), AdCMV-Flagp38(AGF). Equal amounts of total HSP27 could be detected in Ad.vec and Ad.mda-7 infected FO-1 cells. These results further strengthen the involvement of the p38 MAPK pathway in Ad.mda-7-mediated killing of FO-1 cells.

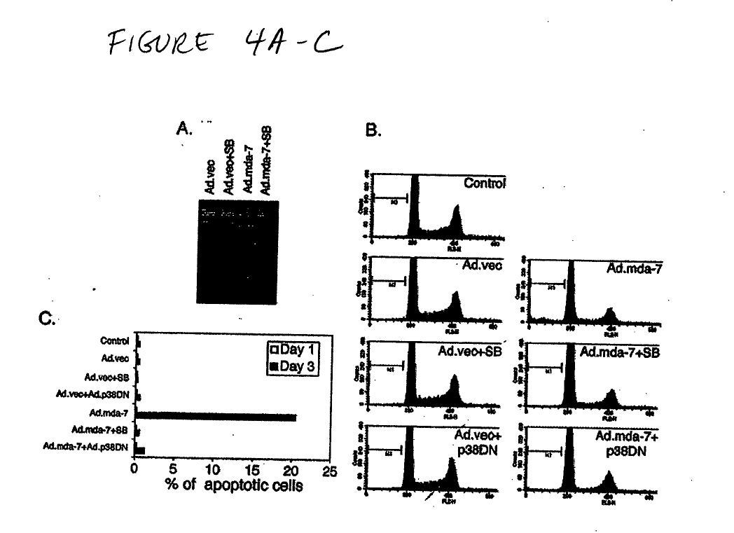

Blocking p38 MAPK Protects Human Melanoma Cells from mda-7-Induced Apoptosis. Experiments were next performed to determine the involvement of the p38 MAPK pathway in regulating cell viability following Ad.mda-7 infection. This was achieved by blocking p38 MAPK pathway pharmacologically with SB203580 or by using AdCMV-Flagp38(AGF). FO-1 cells were infected with Ad.mda-7, alone or with AdCMV-Flagp38(AGF) or they were treated with SB203580 and cell viability was determined using the MTT assay 4 days later. Infection with Ad.mda-7 significantly reduced cell viability (˜75%) 4 days post-infection (FIG. 3), while infection with AdCMV-Flagp38(AGF) or treatment with SB203580 reversed the mda-7 inhibitory effect.

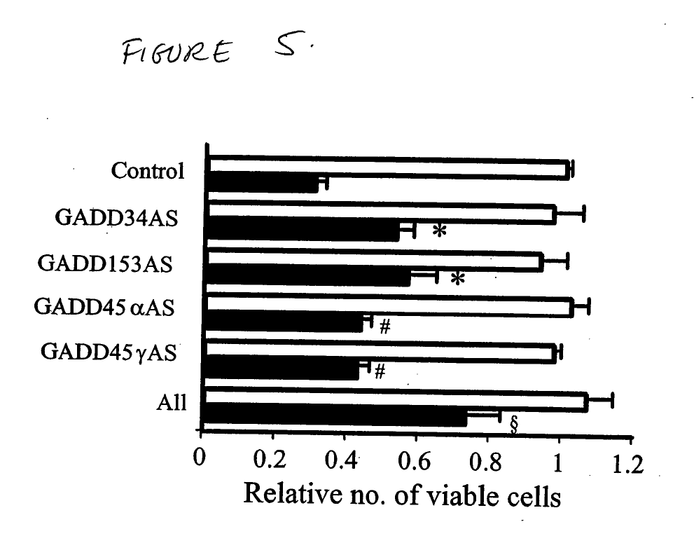

A series of experiments were also performed to determine if SB203580 or AdCMV-Flagp38(AGF) could inhibit Ad.mda-7-mediated apoptosis, as monitored by DNA fragmentation, in FO-1 cells. Three days post-infection of FO-1 cells with Ad.mda-7 resulted in significant DNA fragmentation as compared to control and SB203580-treated cells (FIG. 4A). However, treatment with SB203580 effectively inhibited Ad.mda-7-mediated DNA fragmentation in FO-1 cells. These results were confirmed by cell cycle analysis (FIG. 4B). One day post-infection with Ad.mda-7 no induction of apoptosis was apparent (FIG. 4C). However, at day 3, a significant percentage (˜20%) of cells were apoptotic following Ad.mda-7 infection. Treatment with SB203580 or simultaneous infection with AdCMV-Flagp38(AGF) prevented the induction of apoptosis by Ad.mda-7 (FIGS. 4B and 4C). These results suggest that Ad.mda-7 induces the GADD family of genes via p38 MAPK pathway, which then induce apoptosis.

Inhibition of p38 MAPK Prevents Ad.mda-7 Downregulation of the Anti-Apoptotic Protein BCL-2. Previous studies document that Ad.mda-7 infection results in down-regulation of the anti-apoptotic protein BCL-2 in FO-1 cells (Lebedeva et al., 2002, Oncogene 21, 708-718). It was therefore determined whether SB203580 treatment could counteract this effect of Ad.mda-7 on BCL-2 protein levels. Indeed, as shown in FIG. 2D, treatment with SB203580 prevented Ad.mda-7-mediated downregulation of the BCL-2 protein. This effect of SB203580 was specific for BCL-2 as it did not have any effect on Ad.mda-7-mediated modulation of other pro- or anti-apoptotic proteins.

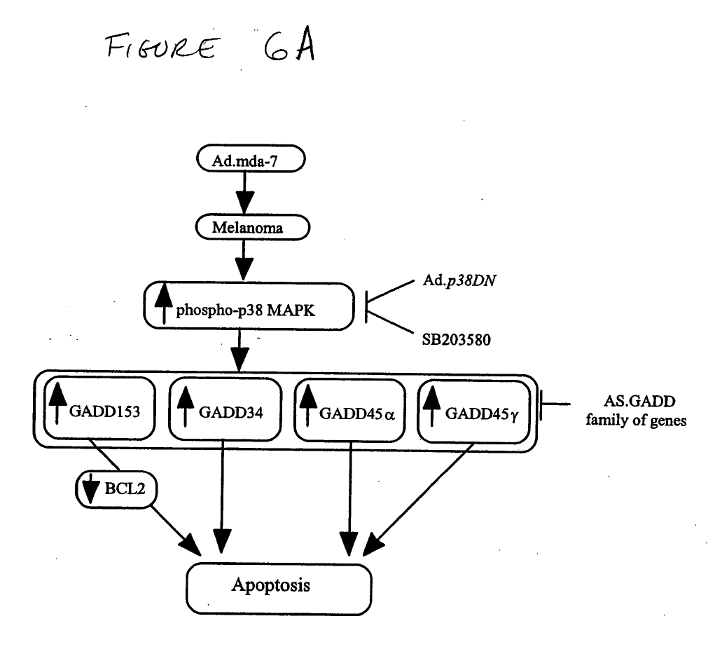

Blocking the GADD Family of Genes Inhibits Ad.mda-7-Induced Apoptosis. An involvement of the GADD family of genes in Ad.mda-7 mediated apoptosis was also confirmed by an anti-sense approach. The different antisense constructs were transfected either alone or in combination into FO-1 cells and then the cells were infected with Ad.vec or Ad.mda-7. Cell growth was analyzed after four days by MTT assay. As shown in FIG. 5, infection with Ad.mda-7 reduced viable cell number by ˜75% as compared to infection with Ad.vec. Transfection of the antisense constructs alone provided small but significant protection against Ad.mda-7 mediated cell death. In contrast, the combination of all of the constructs provided significantly higher protection against Ad.mda-7 mediated cell death, the cell number being reduced by only ˜25% as compared to the Ad.vec-infected cells (FIG. 5). These results indicate that the coordinated overexpression of the GADD family of genes plays an important role in Ad.mda-7 mediated apoptosis.

Discussion

Mda-7/IL-24 holds significant promise for the gene-based therapy of cancers because of its unique capacity to kill cancer cells while sparing normal cells (Su et al., 1998, Proc. Natl. Acad. Sci. USA. 95, 14400-14405; Saeki et al., 2000, Gene Ther. 7, 2051-2057; Su et al., 2001, Proc. Natl. Acad. Sci. USA. 98, 10332-10337; Lebedeva et al., 2002, Oncogene 21, 708-718; Madireddi et al., 2000, Adv. Exp. Med. Biol. 465, 239-261; Mhashilkar et al., 2001, Mol. Med. 7, 271-282; Jiang et al., 1996, Proc. Natl. Acad. Sci. USA. 93, 9160-9165). In this context, this gene is currently undergoing Phase I clinical trials and has been found in an initial study to be safe with only mild toxicities observed and to induce apoptosis in a large percentage of tumor volume when injected intratumorally (Chada et al., 2001, Cancer Gene Ther. 8, S3). However, despite entry into the clinical arena, the molecular mechanism of mda-7 action still remains to be elucidated. The present study is the first to depict a specific signaling pathway responsible for the apoptosis-inducing effect of Ad.mda-7 in the framework of human melanoma cells from which mda-7/IL-24 was first identified and cloned (Jiang et al., 1995, Oncogene 11, 2477-2486).

Two recent reports demonstrate that mda-7/IL-24 can bind and signal through the IL20R1/IL22R2 and IL20R1/IL20R2 receptor complexes and this interaction causes phosphorylation of STAT 3 (Wang et al., 2002, J. Biol. Chem. 277, 7341-7347; Dumoutier et al., 2001, J. Immunol. 167, 3545-3549). However, the biological relevance of STAT 3 phosphorylation is not known. The present study provides direct evidence indicating that the apoptotic effect of Ad.mda-7 in melanoma cells is mediated by the p38 MAPK pathway. There are numerous instances in which activation of the p38 MAPK pathway correlates with induction of apoptosis (Kummer et al., 1997, J. Biol. Chem. 272, 20490-20494; Xia et al., 1995, Science 270, 1326-1331; Juo et al., 1997, Mol. Cell Biol. 17, 24-35; Schwenger et al., 1997, Proc. Natl. Acad. Sci. USA. 94, 2869-2873; Kawasaki et al., 1997, J. Biol. Chem. 272, 18518-18521; Yue et al., 1999, J. Biol. Chem. 274, 1479-1486; Valladares et al., 2000, Endocrinol. 141, 4383-4395). These include the concomitant activation of p38 MAPK and apoptosis induced by diverse agents and experimental conditions, including NGF withdrawal, Fas ligation, and exposure to TNFα, ceramides, sodium salicylates, peroxynitrite and UV radiation (Oh-hashi, et al., 2001, Free Rad. Biol. Med. 30, 213-221; Kummer et al., 1997, J. Biol. Chem. 272, 20490-20494; Xia et al., 1995, Science 270, 1326-1331; Juo et al., 1997, Mol. Cell Biol. 17, 24-35; Schwenger et al., 1997, Proc. Natl. Acad. Sci. USA. 94, 2869-2873; Kawasaki et al., 1997, J. Biol. Chem. 272, 18518-18521; Yue et al., 1999, J. Biol. Chem. 274, 1479-1486; Valladares et al., 2000, Endocrinol. 141, 4383-4395; Galibert et al., 2001, EMBO J. 20, 5022-5031). In addition, the selective p38 MAPK inhibitor, SB203580, can block sodium salicylate-induced FS-4 fibroblast apoptosis, glutamate-induced cerebellar granule cell apoptosis, serum depletion-induced Rat-1 cell death, NGF withdrawal-induced PC12 cell apoptosis, TNFα-mediated rat fetal brown adipocyte apoptosis and TL1-induced bovine pulmonary artery endothelial cell apoptosis (Kummer et al., 1997, J. Biol. Chem. 272, 20490-20494; Schwenger et al., 1997, Proc. Natl. Acad. Sci. USA. 94, 2869-2873; Kawasaki et al., 1997, J. Biol. Chem. 272, 18518-18521; Yue et al., 1999, J. Biol. Chem. 274, 1479-1486). Activation of the GADD family of genes by p38 MAPK might represent a mechanism by which some of these protocols induce apoptosis (Oh-hashi, et al., 2001, Free Rad. Biol. Med. 30, 213-221; Kultz et al., 1998, J. Biol. Chem. 273, 13645-13651; Brenner et al., 1997, J. Biol. Chem. 272, 22173-22181). Fas- or ceramide-induced apoptosis is mediated by p38 MAPK and GADD153 (Brenner et al., 1997, J. Biol. Chem. 272, 22173-22181). Oxidative stress by peroxynitrite induces GADD34, GADD45 and GADD153 via the p38 MAPK pathway and induces apoptosis in neuroblastoma cells (Oh-hashi, et al., 2001, Free Rad. Biol. Med. 30, 213-221).

In this study, the GADD family of genes was shown to be upregulated both at mRNA and protein levels by Ad.mda-7 in human melanoma, but not in normal immortal melanocytes. A similar upregulation of the GADD family of genes (which correlates with apoptosis induction) is observed in glioblastoma multiforme and breast and prostate carcinoma cells, but not in their normal cellular equivalents, following infection with Ad.mda-7. This upregulation in melanoma cells was coupled with the induction of apoptosis and was blocked by SB203580. It has been shown that GADD153 induces its apoptotic effect by downregulating the activity of the bcl-2 promoter (Novoa et al., 2001, J. Cell Biol. 153, 1011-1021). It was previously observed that Ad.mda-7 infection results in downregulation of BCL-2 protein in many different cancer cell types, including melanoma (Su et al., 1998, Proc. Natl. Acad. Sci. USA. 95, 14400-14405; Su et al., 2001, Proc. Natl. Acad. Sci. USA. 98, 10332-10337; Lebedeva et al., 2002, Oncogene 21, 708-718). Based on these observations it was reasoned that if bcl-2 downregulation was caused by Ad.mda-7-mediated induction of GADD153, then treatment with SB203580 should restore the BCL-2 protein to its basal level. Indeed, as shown in FIG. 2D, SB203580 inhibited Ad.mda-7-mediated BCL-2 downregulation, indicating a signaling pathway involving Ad.mda-7, p38 MAPK, GADD153 and bcl-2 (FIG. 6). A role of the GADD family of genes in Ad.mda-7-mediated apoptosis in human melanoma cells is given further credence by the observation that inhibition of the GADD genes, either alone or in combination, counteracted Ad.mda-7 mediated apoptosis. These results indicate that apoptosis induction in human melanoma cells by Ad.mda-7 is mediated by the GADD family of genes, rather than upregulation of the GADD family of genes occurring simply as a consequence of growth arrest and apoptosis.

A very significant question is why Ad.mda-7 fails to elicit any detrimental effects in normal cells. Previous studies document that Ad.mda-7 efficiently infects normal melanocytes and immortal FM516 melanocytes resulting in the production of MDA-7 protein, which is secreted into the medium (Lebedeva et al., 2002, Oncogene 21, 708-718). It does not appear that the FM516 cells are defective in the GADD-induction pathway, since the DNA damaging agent methyl methanesulfonate induces the GADD family of genes in these cells. Infection of FM516 cells with Ad.mda-7 does not result in phosphorylation of p38 MAPK, whereas this effect is observed in FO-1 cells that are induced to undergo apoptosis by mda-7. Although this may partially explain the resistance of FM516 cells to Ad.mda-7, the reason why p38 MAPK is not phosphorylated in these cells is not understood. A recent report has documented that the p38 MAPK pathway is augmented in oncogenically transformed cells, in comparison to normal cells, making these tumor cells sensitive to genotoxic stress (Benhar et al., 2001, Mol. Cell Biol. 21, 6913-6926). This phenomenon might provide an explanation for the resistance of FM516 cells to Ad.mda-7. The basal level of GADD34 mRNA, but not GADD153, expression in FM516 cells was higher than that in the melanoma cell lines (FIG. 1A). It has been shown that GADD34 functions as a negative regulator for the expression of GADD153 during unfolded protein response (Novoa et al., 2001, J. Cell Biol. 153, 1011-1021) providing a possible explanation for the differences in the levels of these two GADD family member genes in FM516 cells. Although both GADD34 and GADD153 are capable of inducing apoptosis either alone or in combination, in certain contexts they might be part of a check and balance loop for viability within the cell. It is possible that the high basal level of GADD34 in FM516 cells might give them some inherent resistance to mda-7-mediated killing.

### EXAMPLE 2

Materials and Methods

Cell lines and culture conditions. HeLa (human cervical cancer), HO-1 (human melanotic metastatic melanoma), and C8161 (human amelanotic melanoma), FM-516 (human melanocytes, immortalized by the SV40 T-antigen), PC-3 and DU-145 (human prostate cancer), hTERT immortalized human fetal astrocytes (Su et al., 2003, Oncogene 22:1164-1180), 2f TGH (human fibrosarcoma) and corresponding mutant sub lines U1A, U3A, U4A and USA (Darnell et al., 1994, Science 264: 1415-1421) were grown in Dulbecco's modified Eagle's medium/F12 (DMEM/F12) supplemented with 10% FBS at 37° C. in a humidified 5% Co₂ incubator. Human pancreatic carcinoma cell lines (BxPC-3, AsPC-1, MIA PaCa-2, and PANC-1) were maintained in RPMI 1640 medium containing 10% FBS, antibiotics and L-glutamine. Rat embryo fibroblast CREF-ras (Su et al., 1993, Oncogene 22:1164-1180; Su et al., 1997, Proc. Natl. Acad. Sci. U.S.A. 94: 9125-9130; Su et al., 2002, J. Cell Physiol. 192: 34-44), normal human breast immortalized epithelial cells HBL-100 and human breast cancer derived lines MCF-7, T47D, MDA-MB-231 and MDA-MB-453 were grown in DMEM containing 10% FBS.

Virus Infection. The mda-7/IL-24 expressing replication defective Ad.mda-7 and corresponding empty adenovirus vector lacking exogenous gene, used as a control (Ad.vector or Ad.vec) has been described previously in Su et al., 1998, Proc. Natl. Acad. Sci. U.S.A. 95: 14400-14405. Stock virus preparations were diluted in DMEM containing 1% FBS and inoculated onto cell monolayers at the indicated multiplicity of infection (MOI). After 2 h virus adsorption at 37° C. with rotary agitation for 15 sec every 15 min, the virus inoculum was removed and DMEM containing 10% FBS was added to the infected monolayer cultures followed by incubation at 37° C. for the indicated times.

MTT Assay. Cells were plated in 96 well dishes (1×10³ cell/well) in DMEM/F12 containing 10% FBS and allowed to attach for 12 h prior to Ad.mda-7 infection (100 or 150 MOI). Treatment with inhibitors was initiated 4 h after infection. During a 5 to 7 day-treatment period, medium was changed twice with fresh inhibitor containing medium at day 3 and 6. Cell growth and viable cell numbers were monitored by 3-(4,5-dimethylthiazol-2-yl)-2,5-diphenyltetrazolium bromide (MTT) staining as described (Lebedeva et al., 2000, 2002). The resulting absorbance measured at 595 nm is directly proportional to the number of viable cells.

Annexin-V binding assay. Cells were trypsinized and washed once with complete media. Aliquots of cells (5×10⁵) were resuspended in complete media (0.5 ml) and stained with FITC-labeled Annexin-V (Oncogene Research Products, Boston, Mass.) according to the manufacturer's instructions. Propidium Iodide (PI) was added to the samples after staining with Annexin-V to exclude late apoptotic and necrotic cells. FACS assay was performed immediately after staining. The percentage of the cells in the apoptotic (Aₒ) fraction was calculated using CellQuest software (Becton Dickinson).

RT-PCR of IL-20R1, IL-20R2 and IL-22R. Total RNA was isolated from 10 cm dishes using guanidine isothiocyanate lysis utilizing the RNAeasy kit (Qiagen Inc. Valencia, Calif.). Reverse transcription was performed on 5μof total RNA with an oligo (dT) primer as previously described. cDNA corresponding to 5 ng of total RNA was amplified for 35 cycles by PCR with specific primers for IL-20R1, IL-20R2 and IL-22R as described (Blumberg et al., 2001, Cell 104: 9-19). The products were analyzed on Ethidium bromide-stained 1% agarose gels following electrophoresis.

Northern blot Analysis. Total RNA was extracted from the cells by using Qiagen RNeasy mini kit according to the manufacturer's protocol, and Northern blotting was performed as described (Su et al., 1997, Proc. Natl. Acad. Sci. U.S.A. 94: 9125-9130). The cDNA probes used were full-length human GADD153, full-length human GADD45-α, β, and γ, a 500-bp fragment from human GADD34, and full-length length human GAPDH.

Results

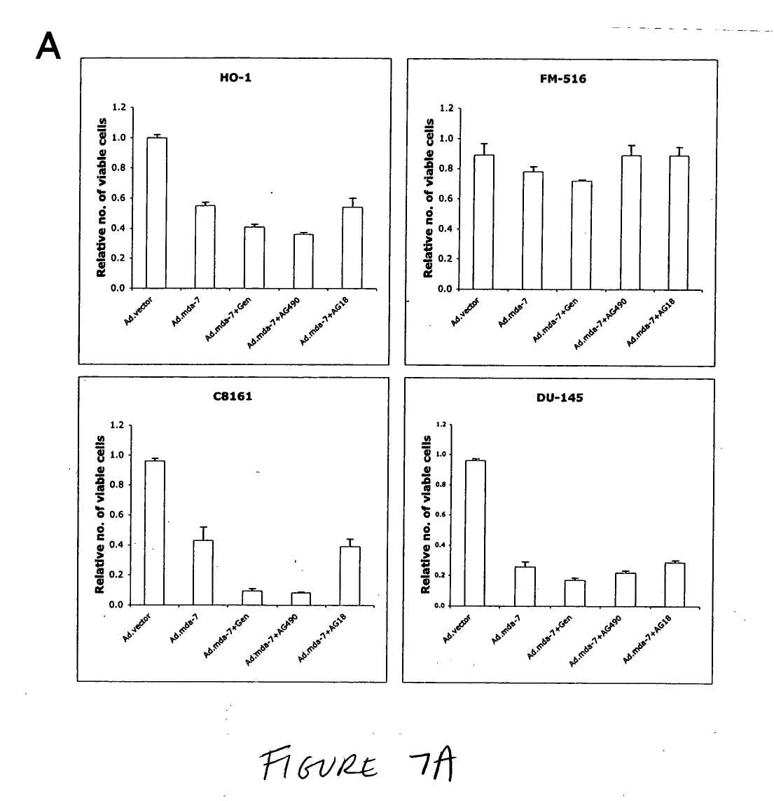

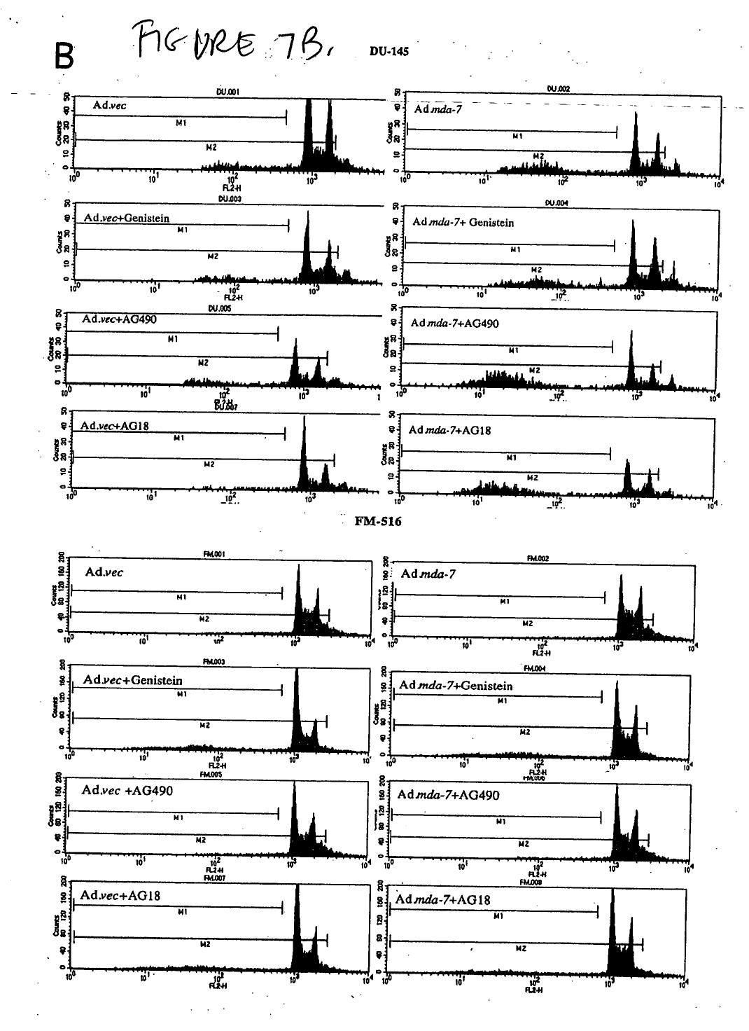

Treatment of cells with TK inhibitors does not prevent mda-7 killing. Two inhibitors of tyrosine kinases, Genistein (Akiyama et al., 1987, J. Biol. Chem. 262: 5592-5595; Williams et al., 2000, Williams et al., 2000, Cytokine 12: 934-943) and tyrphostin AG18 (Gazit et al., 1989, J. Med. Chem. 32: 2344-2352; Catlett-Falcone et al., 1999, Immunity 10: 105-115; Ni et al., 2000, Cancer Res. 60: 1225-1228) were utilized to determine whether tyrosine kinase-mediated phosphorylation is involved in the regulation of Ad.mda-7 induced killing. Whether the JAK-selective inhibitor, AG490, which has been shown to effectively block STAT3 signaling (Catlett-Falcone et al., 1999, Immunity 10: 105-115; Nielsen et al., 1999, Leukemia 13: 735-738; Wang et al., 1999, J. Immunol. 162: 3897-3904; Ni et al., 2000, Cancer Res. 60: 1225-1228) could inhibit Ad.mda-7 cytotoxic activity was also examined based on reports of STAT 3 activation by mda-7/IL24 (Dumoutier et al., 2001, J. Immunol. 167: 3545-3549; Parrish-Novak et al., 2002, J. Biol. Chem. 277: 47517-47523; Pataer et al., 2002, Cancer Res. 62: 2239-2243; Wang et al., 2002, J. Biol. Chem. 277: 7341-7347). Four human cell lines, C8161, DU-145, HO-1 and FM-516 were grown in the absence or presence of inhibitor 4 h post infection. The data shown in FIG. 7A demonstrates that treatment of different cell lines with TK inhibitors, at a concentration that inhibits TK activation, does not interfere with the ability of Ad.mda-7 to kill the susceptible human tumor derived cell lines (C8161, DU-145, HO-1) but as previously reported, does not affect the viability of an immortalized normal human melanocyte (FM-516) (Huang et al., 2001, Oncogene 20: 7051-7063; Lebedeva et al., 2002, Oncogene 21: 708-718). The activity of each inhibitor was separately tested using JAK/STAT responsive reporters to confirm their activity. Ad.mda-7 infection induced a time dependent increase in the proportion of DU-145 cells undergoing apoptosis as reflected by an increase in the proportion of cells with a sub G₀/G₁ (A₀) DNA content (FIG. 7B, DU-145, Ad.mda-7, top panel) as previously described (Madireddi et al., 2000, Adv. Exp. Med. Biol. 465: 239-261; Huang et al., 2001, Oncogene 20: 7051-7063). No overall profile change was observed when inhibitors were present (FIG. 7B Ad.vec and Ad.mda-7 panels with Genistein, AG490 and AG18). Similarly, no significant change in the percentage of apoptotic cells was evident in FM-516 infected with Ad.vector or Ad.mda-7 (FIG. 7B, compare FM-516 to DU-145, panels corresponding to similarly treated cells). Therefore, treatment of susceptible cell lines with Ad.mda-7 in the presence of inhibitors that block JAK/STAT activity does not block cell killing, signifying independence of this activity from the currently described cytokine mediated effects demonstrated by this gene (Dumoutier et al., 2001, J. Immunol. 167: 3545-3549; Caudell et al., 2002, J. Immunol. 168: 6041-6046; Wang et al., 2002, J. Biol. Chem. 277: 7341-7347).

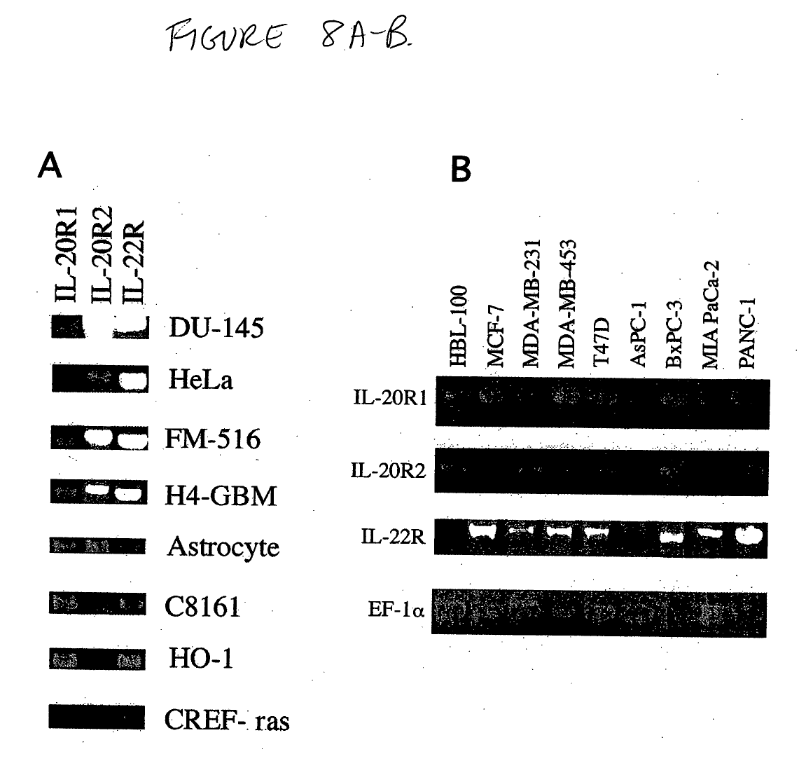

Lack of correlation of IL-20/IL-22 receptor expression with susceptibility to mda-7/IL-24. Recent studies have demonstrated that mda-7/IL-24 can bind the functional heterodimeric IL-20 (IL-20R1/IL-20R2) and IL-22 (IL-22R/IL-20R2) receptor complexes and activate the JAK/STAT signaling cascade, primarily activating STAT3 and to a lesser extent, STAT1 (Parrish-Novak et al., 2002, J. Biol. Chem. 277: 47517-47523; Pataer et al., 2002, Cancer Res. 62: 2239-2243). The expression pattern of IL-20R1, IL-20R2 and IL-22R in susceptible and resistant cells was determined by RT-PCR analysis, expanding findings described previously with normal human tissue derived RNA (Blumberg et al., 2001, Cell 104: 9-19). A panel of resistant, immortalized normal (FIG. 8A, Lanes: Astrocyte and FM-516 melanocytes) as well as susceptible human cancer cell lines (FIG. 8A, Lanes: DU-145, HeLa, H4-GBM, C8161 and HO-1) express the respective receptors. PCR was performed for 35 cycles, which enabled detection of very low expression levels and permitted a determination as to whether expression was either present or absent in a given sample. The results shown are representative of at least three independent RT-PCR reactions for each cell line, with different RNA preparations. Specificity of the reactions was determined by performing RT-PCR on an oncogenic Ras transformed rat embryo fibroblast line (FIG. 8a, Lane CREF-ras), which due to non-conservation of receptor sequences between species, was negative, as demonstrated in other rat fibroblast systems (Zhang et al., 2000, J. Biol. Chem. 275: 24436-24443). In general, it appears that resistance to Ad.mda-7 is independent of receptor expression since both resistant and susceptible lines express detectable levels of receptor MRNA (FIG. 8A, Lanes: Astrocyte and FM-516) and resistance could not be attributed to lack of receptor expression in the cell lines described in FIG. 8A.

To expand the analysis, RT-PCR was performed on a panel of human breast and pancreatic cancer derived cell lines, to determine their receptor expression profile (FIG. 8B). We have previously reported that the immortalized human breast epithelial cell line HBL-100 is resistant to Ad.mda-7 induced killing (Su et al., 1998, Proc. Natl. Acad. Sci. U.S.A. 95: 14400-14405). This line expresses IL-20R1 and IL-20R2 mRNAs (FIG. 8B) as does susceptible lines MCF-7, MDA-MB-231, MDA-MB-453 and T47D (FIG. 8B). The human pancreatic cancer derived lines AsPC-1, BxPC-3, MIA PaCa-2 and PANC-1 present a special case with respect to Ad.mda-7 susceptibility (described below). These lines show a variable expression pattern with respect to receptor MRNA, either not expressing any IL-20R1, IL-20R2 or IL-22R mRNA (AsPC-1), no IL-20R2 (MIA PaCa-2) or expression of all three receptors (BxPC-3 and PANC-1) (FIG. 2B). Conclusions may only be made regarding overall positive or negative receptor gene expression but not about differences in relative levels, as noted earlier.

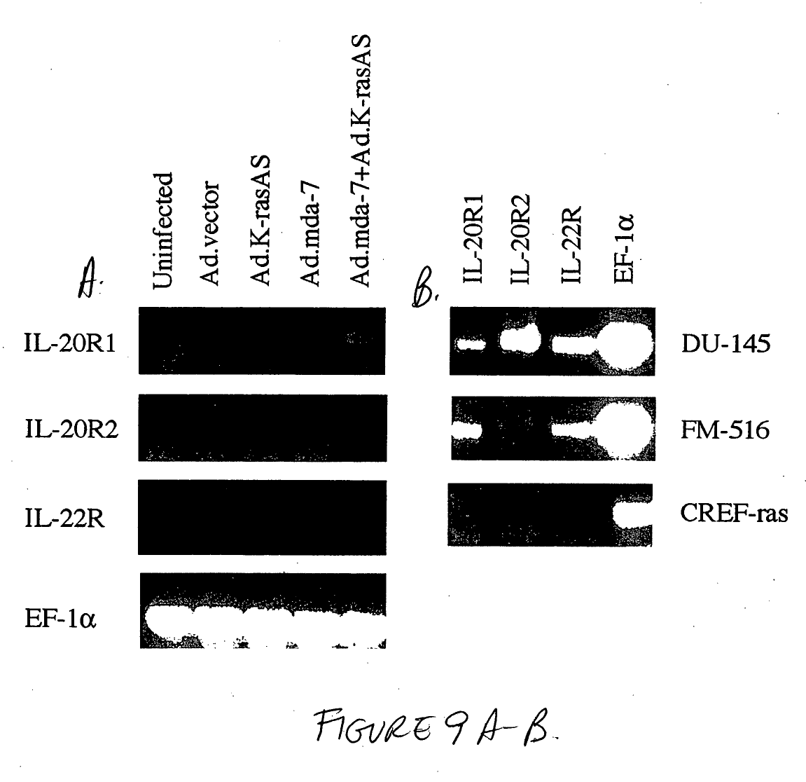

Unlike most other human cancer derived cell lines, infection of pancreatic carcinoma cells with Ad.mda-7 does not significantly alter their growth rate or induce apoptosis (Su et al., 2001, Proc. Natl. Acad. Sci. U.S.A. 98: 10332-10337). However, the combination of Ad.mda-7 with antisense phosphorothioate oligonucleotides or antisense expressing adenovirus, which target the K-ras oncogene (mutated in 85 to 95% of pancreatic carcinomas), induces a dramatic suppression in growth and a decrease in cell viability by induction of apoptosis (Su et al., 2001, Proc. Natl. Acad. Sci. U.S.A. 98: 10332-10337). In mutant K-ras pancreatic carcinoma cells, programmed cell death correlates with expression and an increase in MDA-7/IL-24 and BAX proteins and increases in the ratio of BAX to BCL-2 proteins (Su et al., 2001, Proc. Natl. Acad. Sci. U.S.A. 98: 10332-10337). Since lack of receptor expression was consistently observed in AsPC-1 cells (FIG. 8B), these were utilized to determine if killing of cells occurred as a consequence of receptor upregulation after infection. Infection of AsPC-1 cells with Ad.vector, Ad.mda-7, Ad.K-rasAS or a combination of the latter two viruses, resulted in apparent induction of IL-2081 mRNA, as a generalized response to adenovirus infection compared to uninfected control cells, but continued non-detection of IL-20R2 or IL-22R (FIG. 9A). This result indicated lack of reconstitution or (re-) induction of functional IL-20 or IL-22 receptor complexes under conditions where cells become susceptible to killing. The panel on the right (FIG. 9B) shows DU-145 and FM-516 (positive) and CREF-ras (negative) RNA derived RT-PCR reactions performed in parallel, to control for specificity and authenticity of individual receptor RT-PCR reactions. This observation further emphasizes lack of correlation between susceptibility of a given cell line and pattern of receptor expression since apoptosis (or resistance) occurs irrespective of whether IL-20 or IL-22 receptor gene expression occurs in a given cell type. Lack of availability of specific antibody at the present time has prevented confirmation of these finding at the protein level. However absence of gene expression by sensitive RT-PCR methodology is likely to correlate with non-expression of the corresponding receptor at the protein level.

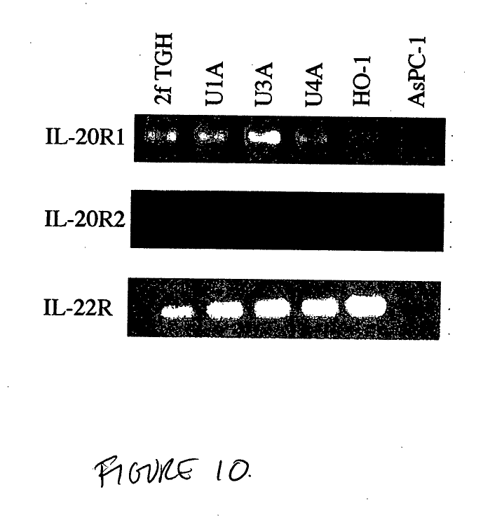

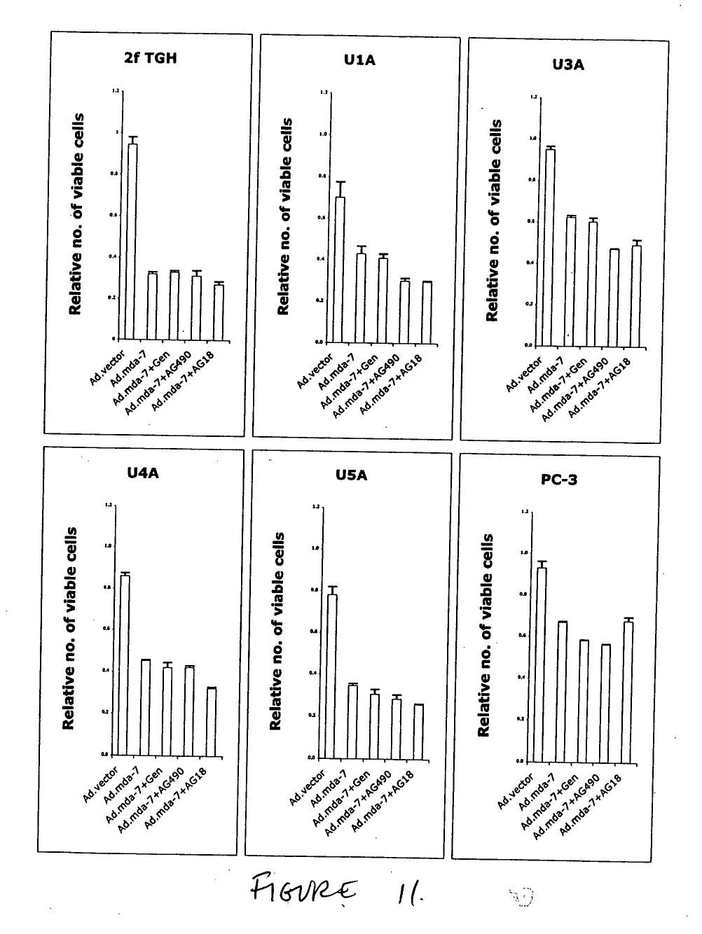

Induction of apoptosis by Ad.mda-7 in mutant cell lines defective in the JAK/STAT pathway. An additional and entirely distinct experimental approach was used to further investigate the specific requirements of the JAK/STAT pathway in Ad.mda-7 mediated killing. Cell lines functionally deficient for JAK/STAT signaling were analyzed for susceptibility to killing. A human fibrosarcoma cell line 2f TGH (parental) and corresponding mutant cell lines derived from it were utilized, including U1A (lacking Tyk2), U3A (IFN-unresponsive, lacking STAT 1), U4A (lacking JAK 1) and USA (lacking IFNAR2c)(kind gift of G. Stark, Cleveland Clinic, Ohio). The human prostate cancer cell PC-3, that does not express STAT3 (Spiotto and Chung, 2000, Prostate 42: 88-98) was used to complete the known spectrum of mda-7/IL-24 mediated pathway components. First, the profile of IL20/IL22 receptor gene expression in the 2f TGH series of cell lines was determined as described above for other cancer types. FIG. 10 demonstrates that all lines have expression of mda-7/IL-24 cognate receptors, indicating that, if cells were resistant to killing, this could not be attributed to lack of receptor expression. These cell lines were infected with Ad.mda-7 and viability analyzed by using a MTT cell proliferation assay. All cell lines were found to be susceptible to Ad.mda-7 (FIG. 11, compare Ad.vector to Ad.mda-7). Ad.mda-7 induced a temporal increase in proportion of 2f TGH, U1A, U3A, U4A, U5A, and PC-3 cells undergoing apoptosis as reflected by an increase in the proportion of cells containing a sub G₀/G₁ (A₀) DNA content (Table 1). In addition, no profile change was observed when inhibitors for the JAK/STAT pathway were present (FIG. 11 and Table 1). In contrast, no significant change in the percentage of apoptotic cells was evident in different cells infected with Ad.vector (FIG. 11 and Table 1). This data provides an independent means of confirmation that the activation of JAK/STAT induced by mda-71/IL-24 (Dumoutier et al., 2001, J. Immunol. 167: 3545-3549; Parrish-Novak et al. J. Biol. Chem. 277: 47517-47523, 2002; Pataer et al., 2002, Cancer Res. 62: 2239-2243; Wang et al., 2002, J. Biol. Chem. 277: 7341-7347) can be separated from cell apoptosis induced after Ad.mda-7 infection since the cells tested have inactivating mutations in JAK/STAT signaling components.

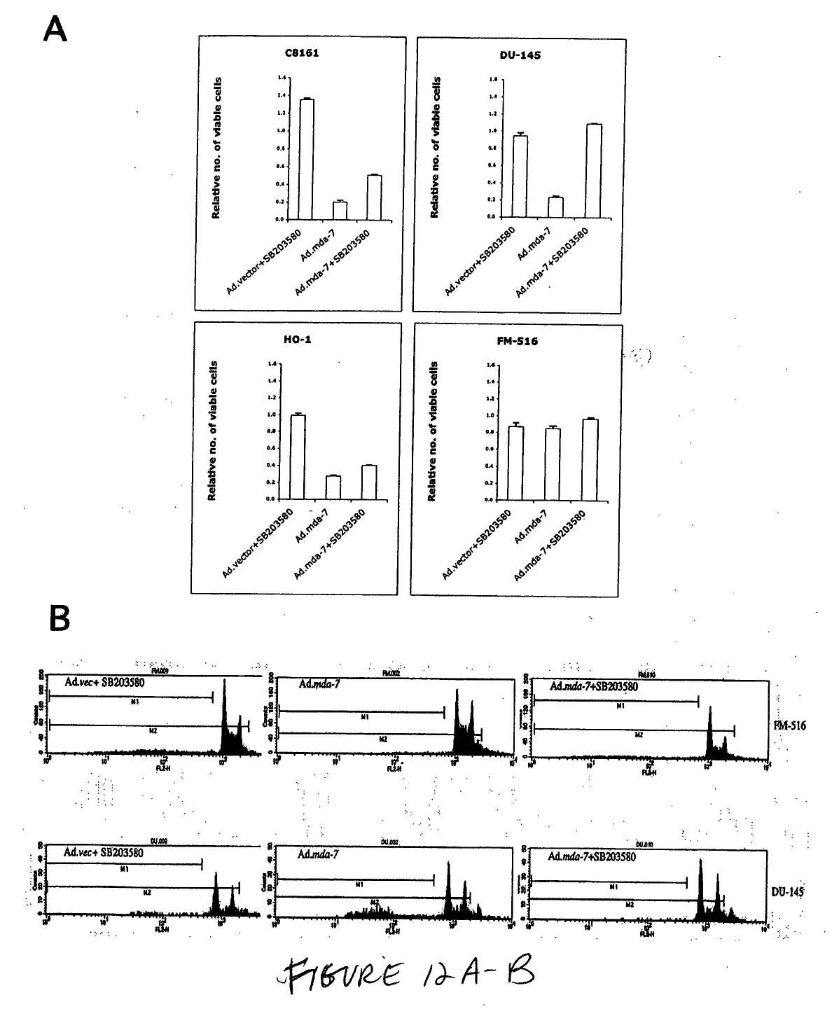

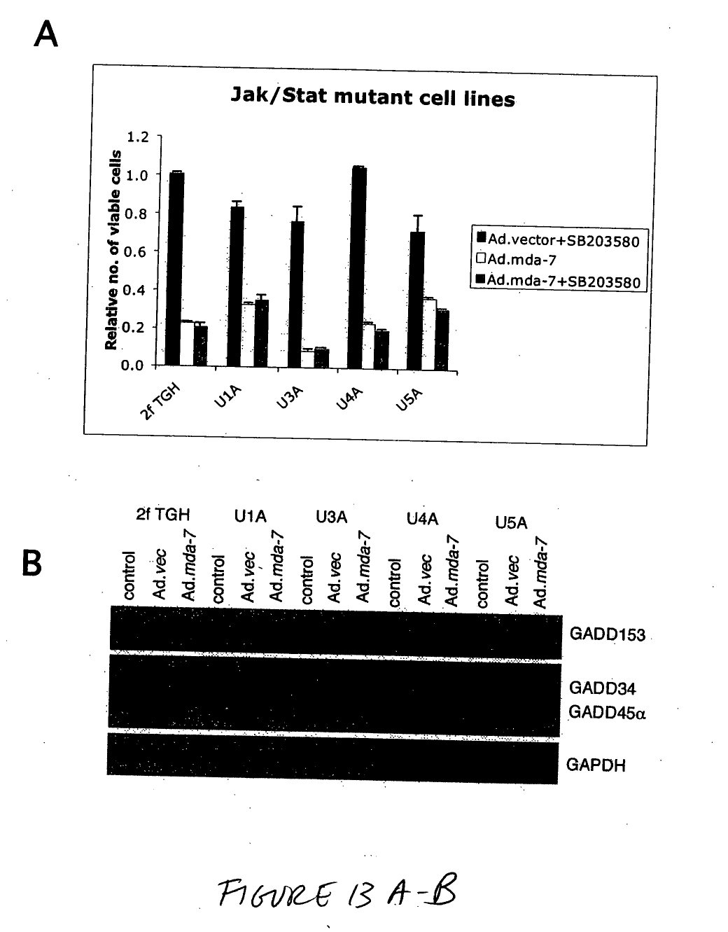

Involvement of p38^{MAPK} in mda-7 mediated apoptosis induction. As discussed in the preceding Section 6, p38^{MAPK} is activated in a melanoma cell line upon Ad.mda-7 infection. It was examined whether p38^{MAPK} plays a role in MDA-7/IL-24 induced killing in a wider spectrum of cell lines using SB203580, a specific p38^{MAPK} signal pathway inhibitor (Davies et al., 2000, Biochem. J. 351: 95-105). Partial inhibition of killing was observed following Ad.mda-7 infection in a melanoma cell line (C8161) and prostate cell lines (PC-3, DU-145) (FIG. 12). The extent of protection varied between each cell line, but some protection was observed in each case. On the other hand, blockage of killing was not obtained with SB203580 in the 2f TGH series of cell lines (FIG. 13A). The absence of SB203580 inhibition of killing 2f TGH and derivate cell lines was correlated with high basal expression levels of GADD family gene expression in the 2f TGH series of cell lines (FIG. 13B). GADD family gene expression was not further enhanced following Ad.mda-7 infection in these cells (FIG. 13B). Based on the observations discussed in Section 6 above, where killing correlated with GADD gene induction, it is possible that cells already expressing relatively high levels of the GADDs have developed resistance to apoptosis induced by these genes. GADD induction is in part p38^{MAPK} dependent and therefore protection by blocking this pathway possibly had no effect in the 2f TGH specific context. Also, more importantly, these results suggest that MDA-7/IL-24 induces apoptosis via induction of multiple pathways in a cell-type dependent manner, as indicated by accumulating reports in the literature (Su et al., 2001, Proc. Natl. Acad. Sci. U.S.A. 98: 10332-10337; Kawabe et al., 2002, Mol. Ther. 6: 637-644; Lebedeva et al., 2002, Oncogene 21: 708-718; Pataer et al., 2002, Cancer Res. 62: 2239-2243; Saeki et al., 2002, Oncogene 21: 4558-4566).

Discussion

Recent experimental evidence documents that the MDA-7 protein can bind to IL20 (IL-20R1/IL-2082) and IL-22 (IL22R/IL-2082) receptor complexes resulting in the activation of JAK/STAT signaling pathways (Kotenko, 2002, Cytokine Growth Factor Rev. 13: 223-240; Sarkar et al., 2002, BioTechniques, Oct. Suppl.: 30-39; Sauane et al., 2003, Cytokine and Growth Factor Reviews 14: 35-51). Engagement of the MDA-7/IL-24 ligand to these receptors was shown to primarily activate STAT3 mediated signaling (Dumoutier et al., 2001, J. Immunol. 167: 3545-3549; Caudell et al., 2002; J. Immunol. 168: 6041-6046 Pataer et al., 2002).

The most frequent clinical disease development correlation associated with JAK/STAT dysregulation involves neoplastic changes (primarily lymphohaematopoietic) and not anti-proliferative, apoptosis inducing effects seen following Ad.mda-7 infection. In particular, STAT3 seems to participate most frequently in the development and maintenance of malignancy. Examples of STAT specific over-activation are seen in multiple myeloma, mycosis fungoides (a T-cell cutaneous lymphoma) and chronic myelogenous leukemia (CML) (Chaff et al., 1997, J. Immunol. 159: 4720-4728; Catlett-Falcone et al., 1999, Immunity 10: 105-115; Nielsen et al., 1999, Leukemia 13: 735-738). STAT3 was also found to be constitutively phosphorylated on tyrosine in mycosis fungoides cells and inhibition of STAT3 DNA binding resulted in induction of apoptosis (Nielsen et al., 1999, Leukemia 13: 735-738). Recent findings demonstrated that constitutive activation of STAT3 occurs frequently in primary prostate adenocarcinomas and is critical for the growth and survival of prostate cancer cells (Mora et al., 2002, Cancer Res. 62: 6659-6666). From these observations, it is difficult to reconcile that Ad.mda-7, which is able to induce STAT3 activity 48 h post-infection, induces apoptosis through this pathway. Accordingly the possibility that there is an inconsistency between cytokine activity related JAK/STAT activation by mda-7/IL24 and its anti-transformed cell activity was pursued in the experiments described in this Section.

The results presented here provide several independent lines of evidence indicating that TK activation is not required for Ad.mda-7 induced apoptosis. In particular, exposure of different cells to two chemically unrelated inhibitors of TK (Genistein and AG18) or JAK-selective inhibitor (AG490) did not prevent Ad.mda-7 induced apoptosis in different cell lines including DU-145, HO-1 and C8161. Similar results were obtained when JAK/STAT deficient cell lines (Darnell et al., 1994, Science 264: 1415-1421) exposed to 150 p.f.u./cell of Ad.mda-7 were demonstrated to be equally susceptible to killing following infection under experimental conditions where immortalized melanocytes (FM-516) and astrocytes were resistant. Following initial confirmation of this unanticipated result, TK or JAK inhibiting capacity of Genistein, AG18 and AG490 respectively was tested using interferon responsive lucifeRase reporter constructs including Type I (IFN-α and β) and Type II/GAS (IFN-γ) to verify that functional inhibitors were being utilized in our studies. Activity of both types of IFN reporters were repressed following treatment with inhibitors for an extended period of exposure (48-72 h) post-transfection, similar to conditions used for Ad.mda-7 activity. This further confirmed that TK activity does not play an integral role in Ad.mda-7 induced killing.

In view of the results obtained with TK inhibitors, a survey for IL-20 and IL-22 receptor gene expression was conducted in several cell lines with known responses to Ad.mda-7 infection. Analysis by RT-PCR of RNA derived from DU-145 (prostate, susceptible), HeLa (cervical, susceptible), FM-516 (immortalized melanocytes, resistant) human fetal astrocytes (resistant), H4 glioma (susceptible) and CREF-ras (immortal cloned rat embryo fibroblast, negative control) using IL-20R1, IL-20R2 and IL-22R specific primers indicated that cell resistance to killing by Ad.mda-7 was not due to lack of appropriate receptor expression. Resistant lines, FM-516 (immortalized melanocytes), BxPC-3 (pancreatic cancer cells) and immortalized human astrocytes, showed receptor MRNA expression patterns similar to that seen in susceptible lines. Detection of receptors in a given cell context does not exclude the possibility that resistance was due to expression of one or more “non-functional” receptor molecules. On the other hand, pancreatic cell lines AsPC-1, MIA PaCa-2 and PANC-1, which are susceptible to killing by Ad.mda-7, only after ablation of endogenous mutated K-ras levels (Su et al., 2001, Proc. Natl. Acad. Sci. U.S.A. 98: 10332-10337), show complete absence of basal levels of functional receptor complex MRNA expression (AsPC-1) or robust expression of all three receptor genes (PANG-1). The possibility of receptor gene induction in the AsPC-1 was examined in a pancreatic line, which did not show basal levels of receptor mRNA expression. Infection by Ad.vector, Ad.mda-7 or combination Ad.mda-7 and antisense K-ras treatment was observed to trigger IL-20R1 mRNA expression after different times post-infection in AsPC-1 cells. This however did not reconstitute functional mda-7/IL-24 receptor (Parrish-Novak et al., 2002, J. Biol. Chem. 277: 47517-47523) but these cells were nevertheless killed subsequent to combination treatment (Su et al., 2001, Proc. Natl. Acad. Sci. U.S.A. 98: 10332-10337). At the other end of the spectrum, the wild type K-ras expressing line BxPC-3 continued to be resistant to killing by Ad.mda-7 in either the presence or absence of the antisense K-ras expressing vector (Su et al., 2001, Proc. Natl. Acad. Sci. U.S.A. 98: 10332-10337) despite apparently robust expression of all three receptor subunits. This survey indicates that the known receptors for mda-7/IL-24 do not appear to play a significant role in the molecule's capacity to induce apoptosis in transformed cells. The data, even in the absence of specific evidence of receptor subunit expression at the protein level is validated by the fact that some cells were susceptible in the absence of detectable levels of one or both receptor chain MRNA by sensitive RT-PCR methods.

An experimentally distinct means of establishing JAK/STAT independence of mda-7/IL-24 mediated killing was achieved by utilizing cells lacking various components of signal transduction pathways. Mutant cell lines including Tyk2 mutant (U1A), STAT1 mutant (U3A), JAK1 mutant (U4A) and deficient STAT3 (PC-3), were used to complete the known spectrum of mda-7/IL-24 mediated pathway components. All cell lines were found to be susceptible to Ad.mda-7. The conclusions of this study were further strengthened by the observations that apoptosis was induced with roughly similar kinetics in the presence or absence of TK inhibitors in all mutant cells lines.

Independent experimental strategies were utilized herein, including (i) effect of specific JAK/STAT and TK inhibitors on cell killing, (ii) determination of receptor gene expression profiles (iii) analysis of JAK/STAT pathway mutated cell lines, to demonstrate that signaling events leading to susceptibility of cancer cells to Ad.mda-7 apoptosis induction is apparently JAK/STAT-independent. These results raise the possibility of the existence of an alternative receptor complex mediating Ad.mda-7 killing that might be only partially dependent and/or completely independent of JAK/STAT transduced mechanisms or that transformed cell killing operates through a receptor independent pathway. A further indirect confirmation of our findings it that Ad.mda-7 infection of different cell lines is documented to trigger several independent pathways. Infection with Ad.mda-7 activates the protein kinase R pathway (Pataer et al., 2002, Cancer Res. 62: 2239-2243), Growth Arrest and DNA Damage inducible (GADD) genes (Section 6, above), and components of the MAPK pathway including JNK (Kawabe et al., 2002, Mol. Ther. 6: 637-644) and p38^{MAPK} (see below). Infection also inhibits angiogenesis (Saeki et al., 2002, Oncogene 21: 4558-4566). These diverse activities appear to be inconsistent with or attributable entirely to potential cytokine related properties of mda-7/IL-24. In addition, the data presented in this Section 7 and Section 6 above demonstrating the ability to partially block killing induced by Ad.mda-7 using a p38^{MAPK} inhibitor as well as resistance of the wild type K-ras pancreatic cell line (BxPC-3), indicates a potentially important role for dysregulated ras signaling events in the activity of mda-7/IL-24 induced killing.

Various publications are cited herein, the contents of which are incorporated by reference herein in their entireties.

TABLE 1

| | control | | Genistein | | AG490 | | AG18 | |
|---|---|---|---|---|---|---|---|---|
| | Ad.vec | Ad.mda-7 | Ad.vec | Ad.mda-7 | Ad.vec | Ad.mda-7 | Ad.vec | Ad.mda-7 |
| PC-3 | 4.96 | 22.1 | 3.73 | 19.10 | 4.17 | 25.34 | 4.88 | 19.27 |
| 2fTGH | 6.93 | 18.67 | 8.14 | 29.85 | 5.98 | 18.56 | 4.64 | 15.64 |
| U1A | 7.06 | 17.73 | 8.03 | 20.83 | 6.18 | 17.13 | 7.64 | 18.79 |
| U3A | 9.21 | 28.26 | 11.67 | 36.29 | 12.84 | 30.52 | 11.76 | 32.95 |
| U4A | 11.32 | 20.89 | 11.97 | 17.94 | 9.35 | 20.32 | 10.47 | 36.14 |
| U5A | 2.74 | 16.65 | 2.69 | 16.69 | 16.25 | 33.08 | 3.77 | 33.43 |

1. A method for identifying an apoptosis modulating agent, comprising:
(a) identifying, as a test cell, a cell which undergoes apoptosis in response to increased expression of a mda-7 gene, where the test cell may be propagated as a substantially homogenous population of test cells;
(b) administering, to a test cell, a test agent; and
(c) determining whether there is, in the test cell, a change in the level of phosphorylation of a molecule selected from the group consisting of p38 MAPK and HSP 27 in response to administration of the test agent;
wherein a change in the level of phosphorylation of a molecule selected from the group consisting of p38 MAPK and HSP27 indicates that the test agent is a modulator of apoptosis.

2. The method of claim 1, further comprising the step of determining whether there is a change in the level of expression of BCL-2, wherein a change in the level of BCL-2 further indicates that the test agent is a modulator of apoptosis.

3. The method of claim 1, further comprising the step of determining whether a change in the level of phosphorylation of p38 MAPK and/or HSP 27 is JAK/STAT independent, wherein a determination that said change is independent of JAK/STAT further indicates that the test agent is a modulator of apoptosis.

4. A method of corroborating the results of claim 1, comprising the further step of determining whether the test agent modulates apoptosis in a test cell comprising exposing the test cell to the test agent and then determining whether apoptosis is occurring.

5. The method of claim 1 wherein the test agent is a small molecule.

6. The method of claim 1 wherein the test agent is a nucleic acid.

7. The method of claim 1 wherein the test agent is a peptide.

8. The method of claim 1 wherein the test agent is a MDA-7 variant.

9. A method for identifying an apoptosis inducing agent, comprising:
(a) identifying, as a test cell, a cell which undergoes apoptosis in response to increased expression of a mda-7 gene, where the test cell may be propagated as a substantially homogenous population of test cells;
(b) administering, to a test cell, a test agent; and
(c) determining whether there is, in the test cell, an increase in the level of phosphorylation of a molecule selected from the group consisting of p38 MAPK and HSP 27 in response to administration of the test agent;
wherein an increase in the level of phosphorylation of a molecule selected from the group consisting of p38 MAPK and HSP27 indicates that the test agent is an inducer of apoptosis.

10. The method of claim 9, further comprising the step of determining whether there is a decrease in the level of expression of BCL-2, wherein a decrease in the level of expression of BCL-2 further indicates that the test agent is an inducer of apoptosis.

11. The method of claim 9, further comprising the step of determining whether a change in the level of phosphorylation of p38 MAPK and/or HSP 27 is JAK/STAT independent, wherein a determination that said change is independent of JAK/STAT further indicates that the test agent is an inducer of apoptosis.

12. A method of corroborating the results of claim 9, comprising the further step of determining whether the test agent induces apoptosis in a test cell.

13. The method of claim 9 wherein the test agent is a small molecule.

14. The method of claim 9 wherein the test agent is a nucleic acid.

15. The method of claim 9 wherein the test agent is a peptide.

16. The method of claim 9 wherein the test agent is a MDA-7 variant.

17. A method for identifying an apoptosis inhibiting agent, comprising:
(a) identifying, as a test cell, a cell which undergoes apoptosis in response to increased expression of a mda-7 gene, where the test cell may be propagated as a substantially homogenous population of test cells;
(b) administering, to a test cell, a test agent; and
(c) determining whether there is, in the test cell, a decrease in the level of phosphorylation of a molecule selected from the group consisting of p38 MAPK and HSP 27 in response to administration of the test agent;
wherein a decrease in the level of phosphorylation of a molecule selected from the group consisting of p38 MAPK and HSP27 indicates that the test agent is an inhibitor of apoptosis.

18. A method for identifying an apoptosis modulating agent, comprising:
(a) identifying, as a test cell, a cell which undergoes apoptosis in response to increased expression of a mda-7 gene, where the test cell may be propagated as a substantially homogenous population of test cells;
(b) administering, to a test cell, a test agent;
(c) determining whether there is, in the test cell, a change in the level of expression of one or more GADD protein selected from GADD153, GADD34, GADD45-alpha. or GADD45-gamma in response to administration of the test agent;
wherein a change in the level of expression of one or more GADD protein indicates that the test agent is a modulator of apoptosis.

19. A method of corroborating the results of claim 18, comprising the further step of administering, to the test cell, a second agent that inhibits phosphorylation of p38 MAPK, such as to determine whether said second agent is able to inhibit a change in the expression level of one or more GADD protein caused by the test agent; wherein the ability of the second agent to inhibit the effect of the test agent indicates that the test agent is a modulator of apoptosis.

20. The method of claim 18, further comprising the step of determining whether there is a change in the level of expression of BCL-2, wherein a change in the level of BCL-2 expression further indicates that the test agent is a modulator of apoptosis.

21. A method of corroborating the results of claim 20, comprising the further step of administering, to the test cell, a second agent that inhibits phosphorylation of p38 MAPK, such as to determine whether said second agent is able to block any change in the expression level of one or more GADD protein and BCL-2 caused by the test agent; wherein the ability of the second agent to block the effects of the test agent indicates that the test agent is a modulator of apoptosis.

22. The method of claim 18, further comprising the step of determining whether a change in the level of expression of one or more GADD protein is JAK/STAT independent, wherein a determination that said change is independent of JAK/STAT further indicates that the test agent is a modulator of apoptosis.

23. A method of corroborating the results of claim 18, comprising the further step of determining whether the test agent modulates apoptosis in a test cell comprising exposing the test cell to the test agent and then determining whether apoptosis is occurring.

24. The method of claim 18 wherein the test agent is a small molecule.

25. The method of claim 18 wherein the test agent is a nucleic acid.

26. The method of claim 18 wherein the test agent is a peptide.

27. The method of claim 8 wherein the test agent is a MDA-7 variant.

28. A method for identifying an apoptosis inducing agent, comprising:
(a) identifying, as a test cell, a cell which undergoes apoptosis in response to increased expression of a mda-7 gene, where the test cell may be propagated as a substantially homogenous population of test cells;
(b) administering, to a test cell, a test agent; and
(c) determining whether there is, in the test cell, an increase in the level of expression of one or more GADD protein selected from GADD153, GADD34, GADD45-alpha. or GADD45-gamma in response to administration of the test agent;
wherein an increase in the level of expression of one or more GADD protein indicates that the test agent is an inducer of apoptosis.

29. A method of corroborating the results of claim 28, comprising the further step of administering, to the test cell, a second agent that inhibits phosphorylation of p38 MAPK, such as to determine whether said second agent is able to inhibit an increase in the expression level of one or more GADD protein caused by the test agent; wherein the ability of the second agent to inhibit the effect of the test agent indicates that the test agent is an inducer of apoptosis.

30. The method of claim 28, further comprising the step of determining whether there is a decrease in the level of expression of BCL-2, wherein a decrease in the level of BCL-2 expression further indicates that the test agent is an inducer of apoptosis.

31. A method of corroborating the results of claim 30, comprising the further step of administering, to the test cell, a second agent that inhibits phosphorylation of p38 MAPK, such as to determine whether said second agent is able to block an increase in the expression level of one or more GADD protein and BCL-2 caused by the test agent; wherein the ability of the second agent to inhibit the effects of the test agent indicates that the test agent is a modulator of apoptosis.

32. The method of claim 28, further comprising the step of determining whether an increase in the level of expression of one or more GADD protein is JAK/STAT independent, wherein a determination that said increase is independent of JAK/STAT further indicates that the test agent is an inducer of apoptosis.

33. A method of corroborating the results of claim 28, comprising the further step of determining whether the test agent induces apoptosis in a test cell comprising exposing the test cell to the test agent and then determining whether apoptosis is occurring.

34. The method of claim 28 wherein the test agent is a small molecule.

35. The method of claim 28 wherein the test agent is a nucleic acid.

36. The method of claim 28 wherein the test agent is a peptide.

37. The method of claim 28 wherein the test agent is a MDA-7 variant.

38. A method for identifying an apoptosis inhibiting agent, comprising:
(a) identifying, as a test cell, a cell which undergoes apoptosis in response to increased expression of a mda-7 gene, where the test cell may be propagated as a substantially homogenous population of test cells;
(b) administering, to a test cell, a test agent; and
(c) determining whether there is, in the test cell, a decrease in the level of expression of one or more GADD protein selected from GADD153, GADD34, GADD45-alpha. or GADD45-gamma in response to administration of the test agent;
wherein a decrease in the level of expression of one or more GADD protein indicates that the test agent is an inhibitor of apoptosis.

39. A method of corroborating the results of claim 38, comprising the further step of determining whether the test agent inhibits apoptosis in a test cell comprising exposing the test cell to the test agent and then determining whether apoptosis is occurring.

40. The method of claim 38 wherein the test agent is a small molecule.

41. The method of claim 38 wherein the test agent is a nucleic acid.

42. The method of claim 38 wherein the test agent is a peptide.

43. The method of claim 38 wherein the test agent is a MDA-7 variant.

44. A method for identifying an apoptosis inducing agent, comprising:
(a). administering, to a test cell a test agent;
(b). determining whether there is, in the test cell, an increase in the level of phosphorylation of a molecule selected from the group consisting of p38 MAPK and HSP 27 in response to administration of the test agent; and
(c). determining whether the result of step (b) depends upon the operation of the JAK/STAT pathway;
wherein an increase in the phosphorylation of p38 MAPK and/or HSP 27, which is independent of the JAK/STAT pathway, indicates that the test agent is an inducer of apoptosis.

45. A method of corroborating the results of claim 44, comprising the further step of determining whether the test agent inhibits apoptosis in a test cell comprising exposing the test cell to the test agent and then determining whether apoptosis is occurring.

46. The method of claim 44 wherein the test agent is a small molecule.

47. The method of claim 44 wherein the test agent is a nucleic acid.

48. The method of claim 44 wherein the test agent is a peptide.

49. The method of claim 44 wherein the test agent is a MDA-7 variant.

50. A method for identifying an apoptosis inducing agent, comprising:
(a). administering, to a test cell a test agent;
(b). determining whether there is, in the test cell, an increase in the level of expression of one or more GADD protein selected from GADD 153, GADD34, GADD45-alpha. or GADD45-gamma in response to administration of the test agent; and
(c). determining whether the result of step (b) depends upon the operation of the JAK/STAT pathway;
wherein an increase in the level of expression of one or more GADD protein, which is independent of the JAK/STAT pathway, indicates that the test agent is an inducer of apoptosis.

51. A method of corroborating the results of claim 50, comprising determining whether an inhibitor of p38 MAPK phosphorylation can inhibit the increase in one or more GADD protein caused by the test agent.

52. A method of corroborating the results of claim 50, comprising the further step of determining whether the test agent inhibits apoptosis in a test cell comprising exposing the test cell to the test agent and then determining whether apoptosis is occurring.

53. The method of claim 50 wherein the test agent is a small molecule.

54. The method of claim 50 wherein the test agent is a nucleic acid.

55. The method of claim 50 wherein the test agent is a peptide.

56. The method of claim 50 wherein the test agent is a MDA-7 variant.

57. A method for identifying an apoptosis inducing agent, comprising:
(a). administering, to a test cell, a test agent;
(b). determining whether there is, in the test cell, an increase in the level of phosphorylation of a molecule selected from the group consisting of p38 MAPK and HSP 27 in response to administration of the test agent;
(c). determining whether there is, in the test cell, an increase in the level of expression of one or more GADD protein selected from GADD153, GADD34, GADD45-alpha. or GADD45-gamma in response to administration of the test agent; and
(d). determining whether the results of steps (b) and (c) depend upon the operation of the JAK/STAT pathway;
wherein an increase in the phosphorylation of p38 MAPK and/or HSP 27 and an increase in the level of expression of one or more GADD protein, which are independent of the JAK/STAT pathway, indicate that the test agent is an inducer of apoptosis.

58. A method of corroborating the results of claim 57, comprising determining whether an inhibitor of p38 MAPK phosphorylation can inhibit the increase in one or more GADD protein caused by the test agent.

59. A method of corroborating the results of claim 57, comprising the further step of determining whether the test agent inhibits apoptosis in a test cell comprising exposing the test cell to the test agent and then determining whether apoptosis is occurring.

60. The method of claim 57 wherein the test agent is a small molecule.

61. The method of claim 57 wherein the test agent is a nucleic acid.

62. The method of claim 57 wherein the test agent is a peptide.

63. The method of claim 57 wherein the test agent is a MDA-7 variant.

64. A method for identifying an apoptosis inducing agent, comprising:
(a). administering, to a test cell, a test agent;
(b). determining whether there is, in the test cell, an increase in the level of phosphorylation of a molecule selected from the group consisting of p38 MAPK and HSP 27 in response to administration of the test agent; and
(c). determining whether there is, in the test cell, an increase in the level of expression of one or more GADD protein selected from GADD 153, GADD34, GADD45-alpha. or GADD45-gamma in response to adtinistration of the test agent; and
wherein an increase in the phosphorylation of p38 MAPK and/or HSP 27 and an increase in the level of expression of one or more GADD protein indicate that the test agent is an inducer of apoptosis.

65. A method of corroborating the results of claim 64, comprising determining whether an inhibitor of p38 MAPK phosphorylation can inhibit the increase in one or more GADD protein caused by the test agent.

66. A method of corroborating the results of claim 64, comprising the further step of determining whether the test agent inhibits apoptosis in a test cell comprising exposing the test cell to the test agent and then determining whether apoptosis is occurring.

67. The method of claim 64 wherein the test agent is a small molecule.

68. The method of claim 64 wherein the test agent is a nucleic acid.

69. The method of claim 64 wherein the test agent is a peptide.

70. The method of claim 64 wherein the test agent is a MDA-7 variant.

71. A method for identifying an apoptosis inducing agent, comprising:
(a) identifying, as a test cell, a cell which undergoes apoptosis in response to increased expression of a mda-7 gene, where the test cell may be propagated as a substantially homogenous population of test cells;
(b). administering, to the test cell, a test agent that decreases the level of BCL-2 expression in the test cell;
(c). administering, to the test cell, prior to, concurrently with, or subsequent to step (b), a second agent that inhibits expression of one or more GADD protein selected from the group consisting of GADD153, GADD34, GADD45-alpha. or GADD45-gamma; and
(d) determining the effect of (b) and (c) on the expression level of BCL-2 in the test cell;
wherein the ability of the second agent to inhibit the decrease in BCL-2 expression due to the test agent indicates that the test agent is an inducer of apoptosis.
